(19) United States
(12) Patent Application Publication
(10) Pub. No.: US 2014/0287455 A1
(43) Pub. Date: Sep. 25, 2014

(54) MICROORGANISM DETECTING DEVICE EVALUATION KIT, MICROORGANISM DETECTING DEVICE EVALUATION KIT MANUFACTURING METHOD, AND MICROORGANISM DETECTING DEVICE EVALUATING METHOD

(71) Applicant: Azbil Corporation, Tokyo (JP)

(72) Inventor: Kanami IRIE, Tokyo (JP)

(73) Assignee: Azbil Corporation, Tokyo (JP)

(21) Appl. No.: 14/220,496

(22) Filed: Mar. 20, 2014

(30) Foreign Application Priority Data
Mar. 22, 2013 (JP) 2013-060201

(51) Int. Cl. C12Q 1/04; C12Q 1/10; C12Q 1/14

(52) U.S. Cl. CPC C12Q 1/04; C12Q 1/14; C12Q 1/10
USPC 435/36; 435/34; 435/38

(57) ABSTRACT

An evaluation kit for a microorganism detecting device includes an immobilized microorganism. A manufacturing method for an evaluation kit for a microorganism detecting device includes immobilizing a microorganism. An evaluating method for the microorganism detecting device includes detecting an immobilized microorganism by the microorganism detecting device. The immobilized microorganisms may be dispersed in a solvent. The solvent may be water. The immobilized microorganisms may be immobilized with an aldehyde. The aldehyde may be formaldehyde. The immobilized microorganisms emit, for example, fluorescent light.

### CROSS REFERENCE TO RELATED APPLICATION

This application claims priority under 35 U.S.C. §119 to Japanese Patent Application No. 2013-060201, filed on Mar. 22, 2013, the entire content of which being hereby incorporated herein by reference.

### FIELD OF TECHNOLOGY

The present invention relates to an environment evaluating technology, and, in particular, relates to an immobilized microorganism and to a microorganism detecting device evaluating method.

In clean rooms, such as bio clean rooms, airborne microorganisms are detected and recorded using microorganism detecting devices. See, for example, Japanese Unexamined Patent Application Publication 2011-83214 and N. Hasegawa, et al., Instantaneous Bioaerosol Detection Technology and Its Application, azbil Technical Review, 2-7, Yamatake Corporation, December 2009. The state of wear of the air-conditioning equipment of the clean room can be ascertained from the result of the microorganism detection. Moreover, a record of microorganism detection within the clean room may be added as reference documentation to the products manufactured within the clean room. Optical microorganism detecting devices draw in air from a clean room, for example, and illuminate the drawn-in air with light. When there is a microorganism included within the air, a microorganism that is illuminated with light emits fluorescence or produces scattered light, enabling detection of the numbers, sizes, and the like, of microorganisms included in the air.

When evaluating the accuracy of a microorganism detecting device, whether or not the microorganism detecting device is detecting correctly the microorganisms that are introduced is tested by introducing, into the microorganism detecting device, live microorganisms of, for example, a known quantity and of a known type. However, intentionally introducing live microorganisms into the microorganism detecting device would contaminate the microorganism detecting device. Moreover, when calibrating a microorganism detecting device using non-microorganism particles, in some cases it will not be possible to evaluate correctly the microorganism detecting device due to differences in the shapes, properties, and the like between microorganisms and non-microorganism particles. Given this, an aspect of the present invention is to provide a microorganism detecting device evaluation kit, a method for manufacturing a microorganism detecting device evaluation kit, and a microorganism detecting device evaluating method wherein a microorganism detecting device can be evaluated safely and accurately.

### SUMMARY

A form of the present invention provides an evaluation kit for a microorganism detecting device, including immobilized microorganisms. Moreover, a form of the present invention provides a method for manufacturing an evaluation kit for a microorganism detecting device, including immobilizing microorganisms. Furthermore, a form of the present invention provides an evaluating method for a microorganism detecting device that includes preparing immobilized microorganisms and detecting the immobilized microorganisms using the microorganism detecting device. The immobilized microorganisms may be dispersed in a solvent. The solvent may be water. The immobilized microorganisms may be immobilized with an aldehyde. The aldehyde may be formaldehyde. The immobilized microorganisms emit, for example, fluorescent light. Note that “fluorescent light” includes autofluorescent light.

The present invention enables the provision of a microorganism detecting device evaluation kit, a method for manufacturing a microorganism detecting device evaluation kit, and a microorganism detecting device evaluating method wherein a microorganism detecting device can be evaluated safely and accurately.

### BRIEF DESCRIPTIONS OF THE DRAWINGS

FIG. 1 is a schematic diagram of a clean room according to an example according to the present invention.

FIG. 2 is a schematic top view diagram of a detecting portion of a microorganism detecting device according to an example according to the present invention.

FIG. 3 is a schematic cross-sectional diagram viewed along the section III-III in FIG. 2, of the detecting portion of the microorganism detecting device according to the example.

FIG. 4 is a graph illustrating the fluorescent intensity for each of various types of microorganisms in an example according to the present invention.

FIG. 5 is a graph illustrating schematically the relationship between the fluorescent intensity and the particle diameters of microorganisms, in an example according to the present invention.

FIG. 6 is a bright field observation image of immobilized Escherichia coli according to an example according to the present invention.

FIG. 7 is a fluorescent observation image of immobilized Escherichia coli according to an example according to the present invention.

FIG. 8 is a fluorescent observation image of immobilized Escherichia coli according to an example according to the present invention.

FIG. 9 is a fluorescent observation image of immobilized Escherichia coli according to an example according to the present invention.

FIG. 10 is a bright field observation image of non-immobilized Escherichia coli according to an example according to the present invention.

FIG. 11 is a fluorescent observation image of non-immobilized Escherichia coli according to an example according to the present invention.

FIG. 12 is a fluorescent observation image of non-immobilized Escherichia coli according to an example according to the present invention.

FIG. 13 is a fluorescent observation image of non-immobilized Escherichia coli according to an example according to the present invention.

FIG. 14 is a bright field observation image of immobilized Staphylococcus epidermidis, according to an example according to the present invention.

FIG. 15 is a fluorescent observation image of immobilized Staphylococcus epidermidis, according to an example according to the present invention.

FIG. 16 is a fluorescent observation image of immobilized Staphylococcus epidermidis, according to an example according to the present invention.

FIG. 17 is a fluorescent observation image of immobilized Staphylococcus epidermidis, according to an example according to the present invention.

FIG. 18 is a bright field observation image of non-immobilized Staphylococcus epidermidis, according to an example according to the present invention.

FIG. 19 is a fluorescent observation image of non-immobilized Staphylococcus epidermidis, according to an example according to the present invention.

FIG. 20 is a fluorescent observation image of non-immobilized Staphylococcus epidermidis, according to an example according to the present invention.

FIG. 21 is a fluorescent observation image of non-immobilized Staphylococcus epidermidis, according to an example according to the present invention.

FIG. 22 is a bright field observation image of immobilized Bacillus subtilis, according to an example according to the present invention.

FIG. 23 is a fluorescent observation image of immobilized Bacillus subtilis, according to an example according to the present invention.

FIG. 24 is a bright field observation image of non-immobilized Bacillus subtilis, according to an example according to the present invention.

FIG. 25 is a fluorescent observation image of non-immobilized Bacillus subtilis, according to an example according to the present invention.

FIG. 26 is a bright field observation image of immobilized Aspergillus niger, according to an example according to the present invention.

FIG. 27 is a fluorescent observation image of immobilized Aspergillus niger, according to an example according to the present invention.

FIG. 28 is a fluorescent observation image of immobilized Aspergillus niger, according to an example according to the present invention.

FIG. 29 is a fluorescent observation image of immobilized Aspergillus niger, according to an example according to the present invention.

FIG. 30 is a bright field observation image of non-immobilized Aspergillus niger, according to an example according to the present invention.

FIG. 31 is a fluorescent observation image of non-immobilized Aspergillus niger, according to an example according to the present invention.

FIG. 32 is a fluorescent observation image of non-immobilized Aspergillus niger, according to an example according to the present invention.

FIG. 33 is a fluorescent observation image of non-immobilized Aspergillus niger, according to an example according to the present invention.

FIG. 34 is a bright field observation image of immobilized Pseudomonas, according to an example according to the present invention.

FIG. 35 is a fluorescent observation image of immobilized Pseudomonas, according to an example according to the present invention.

FIG. 36 is a fluorescent observation image of immobilized Pseudomonas, according to an example according to the present invention.

FIG. 37 is a fluorescent observation image of immobilized Pseudomonas, according to an example according to the present invention.

FIG. 38 is a bright field observation image of non-immobilized Pseudomonas, according to an example according to the present invention.

FIG. 39 is a fluorescent observation image of non-immobilized Pseudomonas, according to an example according to the present invention.

FIG. 40 is a fluorescent observation image of non-immobilized Pseudomonas, according to an example according to the present invention.

FIG. 41 is a fluorescent observation image of non-immobilized Pseudomonas, according to an example according to the present invention.

FIG. 42 is a graph illustrating the relationship between the standardized average fluorescent intensity and the particle diameters of microorganisms, in an example according to the present invention.

### DETAILED DESCRIPTION

An example of the present invention will be described below. In the descriptions of the drawings below, identical or similar components are indicated by identical or similar codes. Note that the diagrams are schematic. Consequently, specific measurements should be evaluated in light of the descriptions below. Furthermore, even within these drawings there may, of course, be portions having differing dimensional relationships and proportions.

The evaluation kit for the microorganism detecting device according to the present example includes an immobilized microorganism. Here immobilized microorganisms are microorganisms that are immobilized through, for example, cross-linking of proteins by an immobilizing agents. The immobilizing agent may use an aldehyde such as, for example, formaldehyde or glutaraldehyde.

Examples of immobilized microorganisms include bacteria and fungi. Gram-negative bacteria and Gram-positive bacteria can be listed as examples of bacteria. Escherichia coli, for example, can be listed as an example of a Gram-negative bacterium. Staphylococcus epidermidis, Bacillus atrophaeus, Micrococcus lylae, and Corynebacterium afermentans can be listed as examples of Gram-positive bacteria. Aspergillus species such as Aspergillus niger can be listed as examples of fungi. Note, however, that the immobilized microorganisms are not limited to these specific examples.

The immobilized microorganisms are essentially inactive or dead, and lack the ability to undergo fission and propagate.

The immobilized microorganisms are dispersed, for example, into a solvent such as water. The present inventor discovered that microorganisms, even when immobilized, disperse without agglomerating. The present inventor discovered that immobilized microorganisms fluoresce essentially identically to prior to immobilization. For example, the microorganism detecting device can use the light scattering characteristics that are dependent on the size, such as the particle diameter, of a particle, the intensity of fluorescence emitted by the particle, the wavelength of the fluorescent light emitted by the particle, and the like, to determine whether or not a particle is a microorganism, and if the particle is a microorganism, to determine the type of microorganism. Because immobilized microorganisms do not agglomerate, they are essentially of the same size as prior to immobilization, and emit essentially the same fluorescence as prior to immobilization, it is possible to use immobilized microorganisms to perform the evaluation without contaminating the microorganism detecting device. Moreover, immobilized microorganisms, because they are immobilized, have durability, and are easily handled.

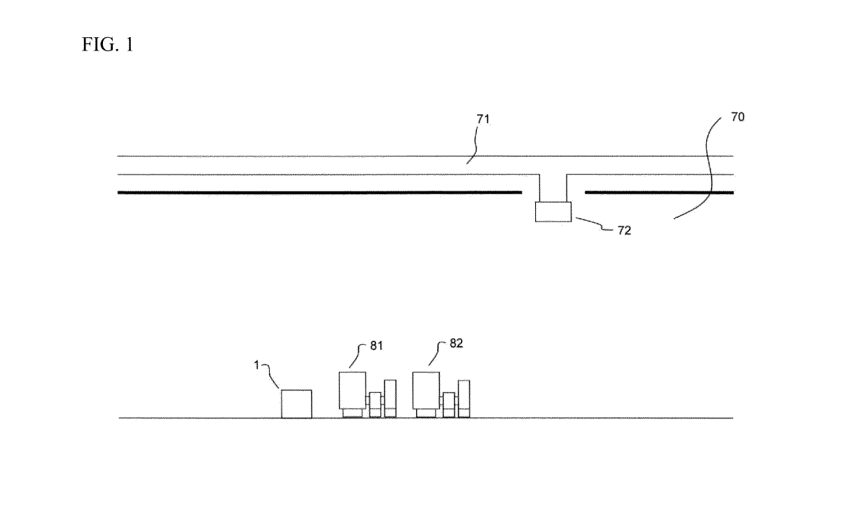

An example of a microorganism detecting device that is evaluated by a microorganism detecting device evaluation kit according to an example will be explained next. As illustrated in FIG. 1, a microorganism detecting device 1 is disposed in, for example, a clean room 70. In the clean room 70, clean air is blown in through a duct 71 and through a blowing opening 72 having an ultrahigh performance air filter such as a HEPA filter (High Efficiency Particulate Air Filter) or ULPA filter (Ultra Low Penetration Air Filter), or the like.

Manufacturing lines 81 and 82 are arranged inside of the clean room 70. The manufacturing lines 81 and 82 are manufacturing lines, for, for example, precision instruments, electronic components, or semiconductor devices. Conversely, the manufacturing lines 81 and 82 may be manufacturing lines for foodstuffs, beverages, or pharmaceuticals. For example, in the manufacturing lines 81 and 82, an infusion liquid may be filled into an intravenous infusion device or a hypodermic. Conversely, the manufacturing lines 81 and 82 may manufacture oral medications or Chinese herb medications. On the other hand, the manufacturing lines 81 and 82 may fill containers with a vitamin drink or beer.

The manufacturing lines 81 and 82 normally are controlled so that microorganisms and non-microorganism particles, and the like, are not dispersed into the air within the clean room 70. However, manufacturing lines 81 and 82, for some reason, are sources that produce microorganisms and non-microorganism particles that become airborne in the clean room 70. Moreover, factors other than the manufacturing lines 81 and 82 also disperse microorganisms and non-microorganism particles into the air of the clean room 70.

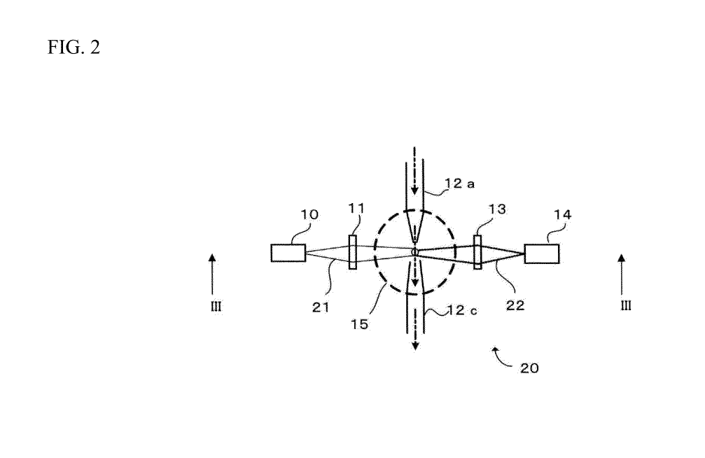

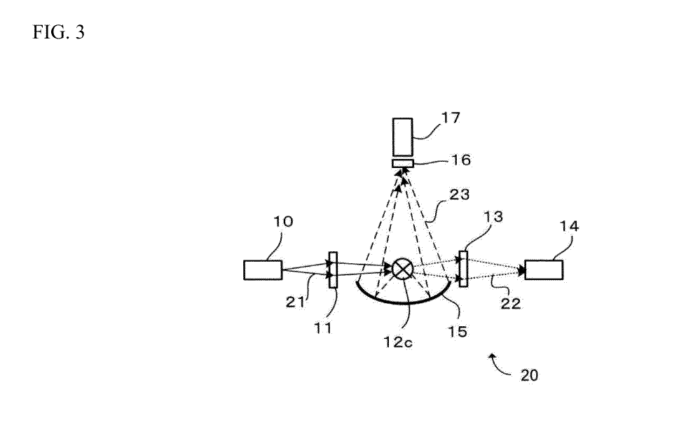

The microorganism detecting device 1 is provided with a detecting portion 20 that is illustrated in FIG. 2 and FIG. 3. The detecting portion 20 is provided with a light source 10, a focusing lens 11 for condensing the light that is emitted by the light source 10, a test sample flow path 12a that includes a nozzle for spraying air, drawn in from the clean room 70, toward the focal point of the focusing lens 11, and a test sample flow path 12c into which is introduced the gas that is sprayed from the test sample flow path 12a. The gas is caused to flow into the test sample flow path 12c from the test sample flow path 12a, illustrated in FIG. 2, at a constant flow rate by the exhaust fan and a pressure regulator, and the like.

A solid-state laser, a gas laser, a semiconductor laser, a light-emitting diode, or the like, can be used as the light source 10. Where a particle is included in the gas that is sprayed from the test sample flow path 12a, the particle, when illuminated with the light, produces scattered light. The scattered light is focused by a focusing lens 13 to be detected by a scattered light detecting device 14. A photodiode, or the like, may be used as the scattered light detecting device 14. When scattered light impinges on the scattered light detecting device 14, an electrical scattered light detection signal is produced and sent to a processing portion, such as a computer system.

The processing portion evaluates whether or not there is a particle based on whether or not there is a scattered light detection signal. Moreover, the processing portion counts the number of particles based on the number of times that a scattered light detection signal has been received. Moreover, because there is a correlation between the intensity of the scattered light and the size of the particle, the processing portion calculates the size, such as the diameter, of the detected particle based on the intensity of the scattered light. Moreover, if the particle is a microorganism, the size of the microorganism will be different depending on the type of microorganism. Because of this, the processing portion may identify the type of microorganism from the size that is calculated.

Moreover, when a fluorescent particle, such as a microorganism, is included in the gas that is sprayed from the test sample flow path 12a, the particle, when illuminated with light, will emit fluorescent light. For example, riboflavin, flavin nucleotides (FMN), flavin adenine dinucleotide (FAD), nicotinamide adenine dinucleotide phosphate (NAD(P)H), pyridoxamine, pyridoxal phosphate (pyridoxal-5′-phosphate), pyridoxine, tryptophan, tyrosine, phenylalanine, and the like, that are included in the microorganisms will produce fluorescence. The fluorescent light is focused by a focusing mirror 15 and detected by the fluorescent light detecting device 17 illustrated in FIG. 3. A filter 16 for shielding the fluorescent light detecting device 17 from the light of wavelengths other than those of the fluorescent light may be disposed in front of the fluorescent light detecting device 17. A photodiode, or the like, may be used for the fluorescent light detecting device 17. When fluorescent light is received by the fluorescent light detecting device 17, an electrical fluorescent light detection signal is produced and sent to a processing portion, such as a computer system.

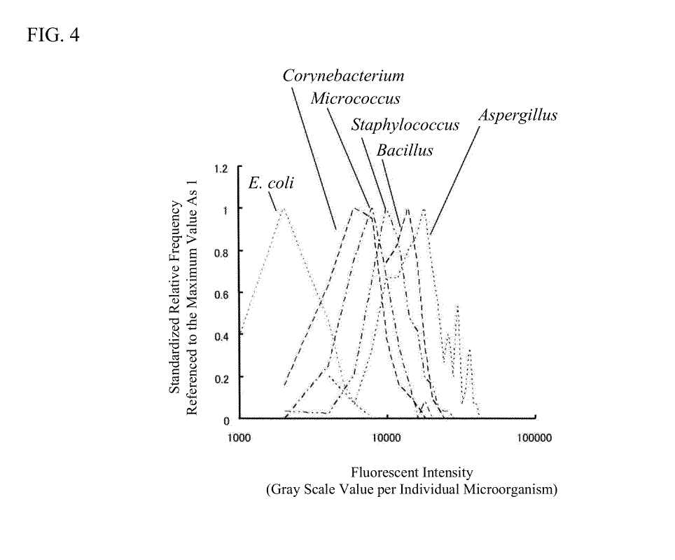

The processing portion evaluates whether or not there is a fluorescent particle based on whether or not there is a fluorescent light detection signal. Moreover, the processing portion counts the number of fluorescent particles based on the number of times that a fluorescent light detection signal has been received. Moreover, as illustrated in FIG. 4, the intensity of the fluorescent light that is produced by the microorganism will differ depending on the type of microorganism. Because of this, the processing portion may identify the type of microorganism from the intensity of the detected fluorescent light.

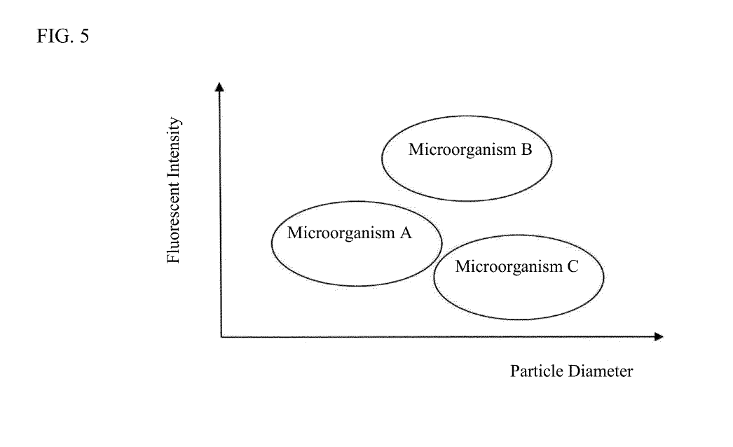

Moreover, when, for example, the processing portion detects scattered light and fluorescent light, it may evaluate the detected particle as a microorganism particle. Moreover, when the processing portion detects scattered light but does not detect fluorescent light, it may evaluate the detected particle as a non-microorganism particle. Moreover, the processing portion, although not limited thereto, may identify the type of microorganism based on both the fluorescent light intensity and the scattered light intensity following the method disclosed in U.S. Pat. No. 6,885,440 and U.S. Pat. No. 7,106,442. For example, as illustrated in FIG. 5, correlations can be seen with the particle diameters and fluorescent intensities depending on the type of microorganism. Consequently, it is possible to identify the type of microorganism from the fluorescent intensity and the particle diameter through obtaining in advance a graph such as illustrated in FIG. 5.

The microorganism detecting device 1 may also identify the type of microorganism by detecting the microorganism through scattered light alone, without detecting fluorescent light. It is possible to identify the type of microorganism through a statistical method such as, for example, the support vector machine (SVM) method by using concentric circular scattered light detecting devices and calculating the scattered light intensity for individual angles. (See, for example, Murugesan Venkatapathi et al., “High-speed Classification of Individual Bacterial Cells Using a Model-based Light Scatter System and Multivariate Statistics,” Applied Optics, USA, Optical Society of America, Feb. 10, 2008, Vol.v47, No. 5, pp. 678 through 686.)

Conversely, the microorganism detecting device 1 may detect the fluorescent spectrum when excitation beams of a plurality of wavelengths are directed toward the gas. For example, it is possible to identify a microorganism based on the detection of the florescent spectrum at wavelengths of 350 nm, 450 nm, and 550 nm, when a microorganism is illuminated with an excitation beam with wavelengths of 266 nm and 355. (See, for example, Vasanthi Sivaprakasam et al., “Multiple UV wavelength Excitation and Fluorescence of Bioarerosols,” Optics Express, USA, Optical Society of America, Sep. 20, 2004, Vol. 12, No. 19, pp. 4457 through 4466.)

When the microorganism detecting device 1 illustrated in FIG. 1 has detected a microorganism, a cleaning operation, or the like, is then performed in the clean room 70.

To evaluate accurately the microorganism detecting capability of the microorganism detecting device 1, preferably microorganisms of a known type and a known quantity are introduced into the microorganism detecting device 1, to test whether or not the types and quantities of microorganisms detected by the microorganism detecting device 1 are the same as those that have been introduced. However, live microorganisms are sensitive to changes in the environment, and sometimes the characteristics thereof change after introduction into the microorganism detecting device 1 from before. Moreover, the introduction, into the microorganism detecting device 1 that is being evaluated, of live called microorganisms that can propagate becomes a source of contamination for the microorganism detecting device 1 and the clean room 70. In particular, because mold, and the like, is readily dispersed in the air, there is a tendency for it to be a powerful contaminant.

Given this, in the past evaluating a microorganism detecting device 1 through introducing non-microorganism particles into the microorganism detecting device 1 has been proposed. However, sometimes there are differences in index of refraction, size, and the like, between non-microorganism particles and actual microorganisms, and in such cases the characteristics of the scattered light that is produced will be different. Moreover, the intensities and wavelengths of the fluorescent light are also different for the actual microorganisms from that of the fluorescent non-microorganism particles. Because of this, depending on the fluorescent non-microorganism particle, there will be cases wherein the microorganism detecting capability of the microorganism detecting device 1 cannot be evaluated accurately. Moreover, when it comes to the size and fluorescence characteristics, even though there are non-microorganism particles that are similar to specific microorganisms, there are also cases wherein it is difficult to prepare non-microorganism particles that have similar characteristics to each of the plurality of types of microorganisms.

In contrast, the immobilized microorganisms included in the microorganism detecting device evaluation kit according to the present example are essentially inactive and dead, and lack the ability to undergo fission or propagate. Because of this, the degree to which the immobilized microorganisms cause contamination to the microorganism detecting device 1 and the clean room 70 is limited. Moreover, because the immobilized microorganisms are essentially the same size as prior to immobilization and emit fluorescence that is essentially identical to that of prior to immobilization, it is possible to evaluate the microorganism detecting device 1 safely and accurately through introducing, into the microorganism detecting device 1, specific types and specific quantities of immobilized microorganisms. Moreover, because the fluorescent attenuation characteristics of the immobilized microorganisms are identical to what they were prior to immobilization, they can be used also in evaluating a microorganism detecting device that detects microorganisms based on the attenuation of florescent light.

When introducing the immobilized microorganisms into the microorganism detecting device 1, the immobilized microorganisms may be dispersed into a gas and the gas that includes the immobilized microorganisms that have been dispersed may be introduced into the microorganism detecting device 1. Conversely, a solution may be prepared through dispersing the immobilized microorganisms into a solvent, such as water, and then producing an aerosol that includes the immobilized microorganisms from that fluid using a nebulizer, or the like, and then introducing it into the microorganism detecting device 1. Because the immobilized microorganisms are cross-linked, there is a tendency for the structure to thereof to be resistant to break down, even when there is a change in the environment. If the microorganism detecting device is able to detect microorganisms in a liquid, a liquid that includes the immobilized microorganisms may be introduced into the microorganism detecting device.

### Examples

Examples of the present invention will be described below. Note, however, that the present invention is, of course, not limited to the examples below.

### Preparing the Immobilized Samples

Formaldehyde solutions with concentrations of 8% and 16% were prepared by diluting a 37% formaldehyde (HCHO) solution (Sigma-Aldrich Japan Co., Ltd., 252549-25ML) in sterilized water.

### Obtaining the Microorganisms

The microorganisms obtained were Escherichia coli (abbreviated “E. coli,” ATCC 13706), Staphylococcus epidermidis (abbreviated “S. epidermidis,” ATCC 12228), Bacillus atrophaeus (abbreviated “B. atrophaeus,” ATCC 9372), Aspergillus niger (abbreviated “A. niger,” ATCC 9142), and Pseudomonas putida (abbreviated “P. putida,” ATCC 12633). Note that ATCC is an abbreviation for the “American Type Culture Collection.” The E. coli and P. putida are Gram-negative bacteria. The S. epidermidis, and B. atrophaeus are Gram-positive bacteria. The A. niger is a fungus. Note that, for the B. atrophaeus, that which is sold as a suspension already was purchased.

### Preparing the Microorganisms

The bacterial microorganisms obtained, aside from the B. atrophaeus and the A. niger cultured through inoculation into a Trypticase soy broth (TSB) medium and cultured overnight while shaking in an isothermal layer at about 32° C. Following this, the bacterial culture was streaked into a pore medium R tryptic soy agar (TSA) medium, and cultivated for approximately 24 hours in an isothermal layer at approximately 32° C. Thereafter, the bacteria were scraped out and suspended in sterilized water. After centrifuging this suspension for three minutes at 2100 g, the supernatant liquid was removed and a pellet was suspended in sterilized water, to obtain a microorganism suspension.

The A. niger, which is a fungus, is caused to produce spores in an agar plate, and a dioctyl sodium sulfosuccinate solution was poured therein, and the spores were dispersed in the liquid. Following this, after recovering the liquid that includes the spores, the spores were removed through filtration through sterilized gauze, and then centrifuged for another 10 minutes at between 1500 g and 1600 g, and the separated pellet was suspended in sterilized water, with this procedure repeated three times, to produce a microorganism suspension.

### Preparing the Immobilized Microorganisms

For the S. epidermidis and the P. putida, a formaldehyde solution with a concentration of 8% and the microorganism suspension were mixed at a 1:1 volume ratio. Specifically, a formaldehyde solution with a volume of 400 μL and a concentration of 8% and the microorganism suspension, with the volume of 400 μL were mixed.

For the E. coli, the B. atrophaeus, and the A. niger, a formaldehyde solution with a concentration of 16% and the microorganism suspension were mixed at a 1:1 volume ratio. Specifically, a formaldehyde solution with a volume of 400 μL and a concentration of 16% and the microorganism suspension, with the volume of 400 μL were mixed.

After mixing, light pipetting was performed, and for the case of E. coli, the sample was allowed to sit for 10 minutes at room temperature. In the cases of S. epidermidis and P. putida the sample was allowed to sit for 5 minutes at room temperature. In the case of the B. atrophaeus, the sample was allowed to sit for 24 hours at room temperature. In the case of A. niger the sample was allowed to sit for 10 minutes at room temperature. Thereafter, each mixed solution was centrifuged for three minutes at 4700 rpm, and the separated pellet was suspended in sterilized water, and this procedure was repeated twice, to produce the sample liquids wherein the immobilized microorganisms are dispersed in sanitized water as the medium.

### Preparing the Control

Sterilized water and the microorganism suspension were mixed at a 1:1 volume ratio. Specifically, sterilized water with a volume of 400 μL, and the microorganism suspension, with the volume of 400 μL, were mixed. After mixing, light pipetting was performed, and for the case of E. coli, the sample was allowed to sit for 10 minutes at room temperature. In the cases of S. epidermidis and P. putida the sample was allowed to sit for 5 minutes at room temperature. In the case of the B. atrophaeus, the sample was allowed to sit for 24 hours at room temperature. In the case of A. niger the sample was allowed to sit for 10 minutes at room temperature. Thereafter, each mixed solution was centrifuged for three minutes at 4700 rpm, and the separated pellet was suspended in sterilized water, and this procedure was repeated twice, to produce the control liquids wherein the non-immobilized microorganisms are dispersed in sanitized water as the medium.

### Microscopic Observations

1.5 μL of each of the sample solutions and the control solutions were dripped onto the slide glasses and dried. Thereafter, a microscope (Olympus BX51) that was provided with an excitation beam filter (Olympus NV BA455) was used to make bright field observations and fluorescent observations of the immobilized microorganisms and the non-immobilized microorganisms.

Results of the Observations of the Immobilized E. coli

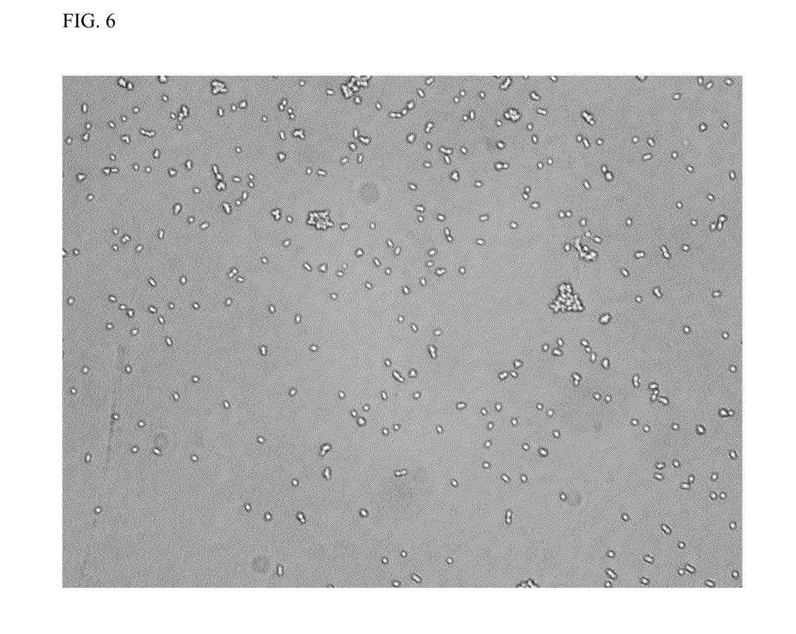

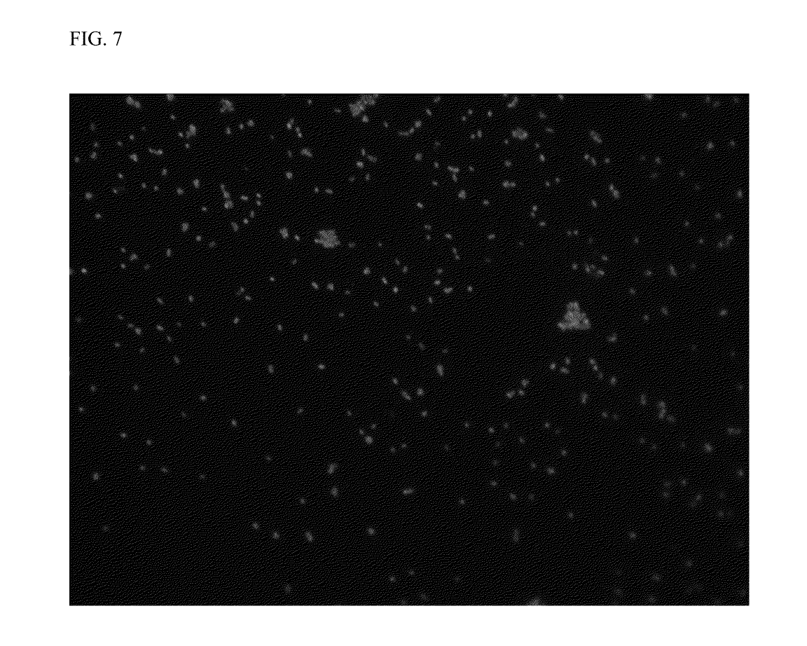

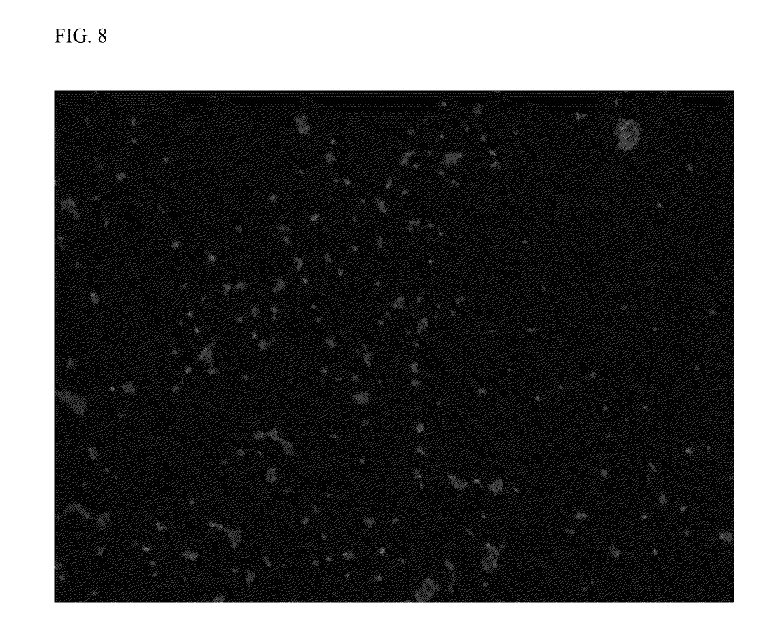

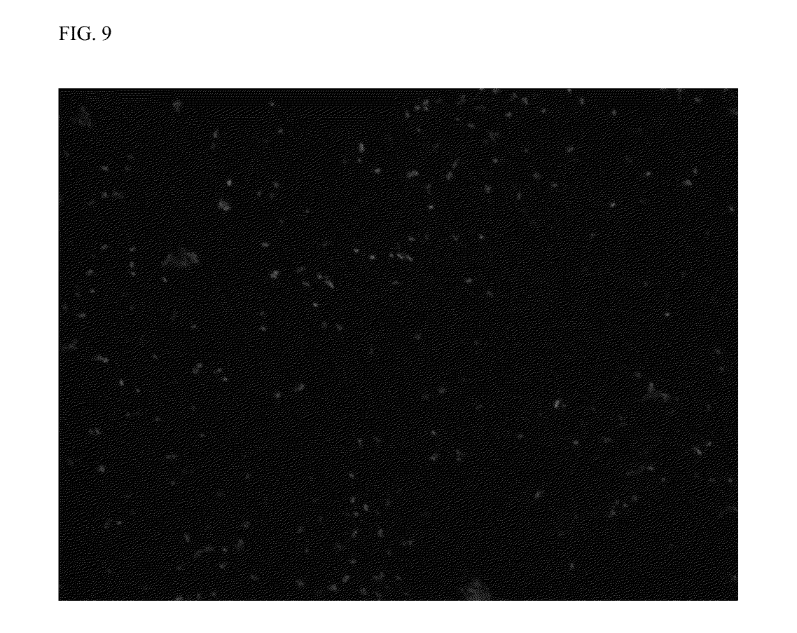

FIG. 6 shows a bright field observation picture of immobilized E. coli, observed immediately after preparation of the sample solution. Additionally, FIG. 7 shows a fluorescent observation picture of immobilized E. coli, observed immediately after preparation of the sample solution. In addition, FIG. 8 shows a fluorescent observation picture of the immobilized E. coli observed after storage for about one day in a cool dark place after preparation of the sample solution, and FIG. 9 shows a fluorescent observation picture of the immobilized E. coli observed after storage for about five days in a cool dark place after preparation of the sample solution. Note that the images in FIG. 7, FIG. 8, and FIG. 9 are images that were captured with an exposure time of 1.0 seconds.

Results of the Observations of the Non-Immobilized E. coli

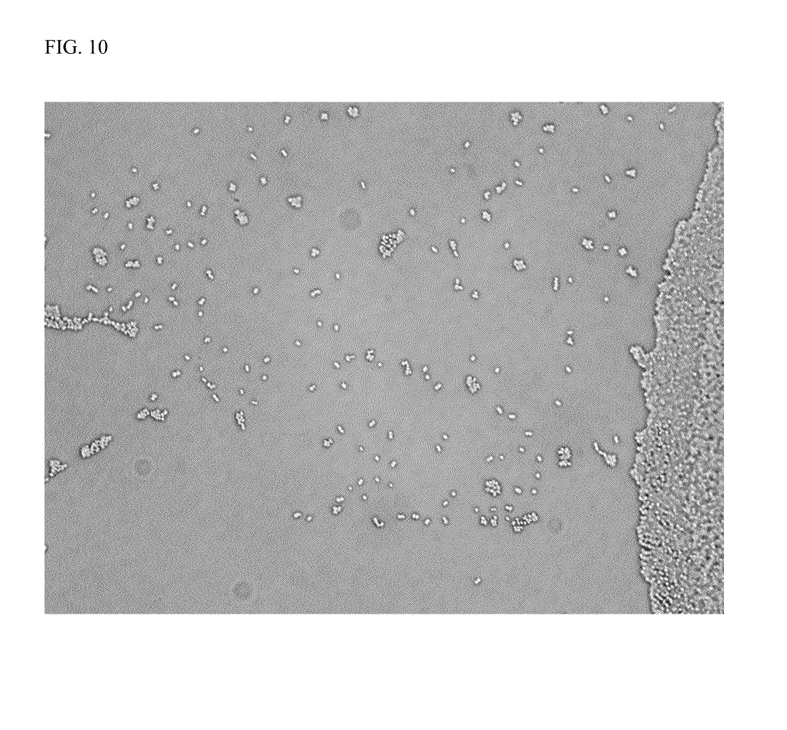

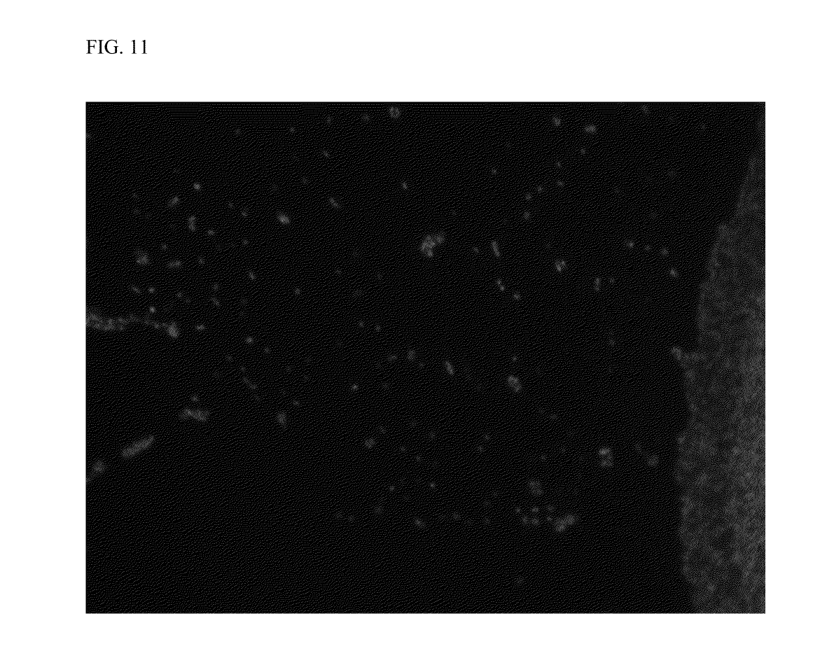

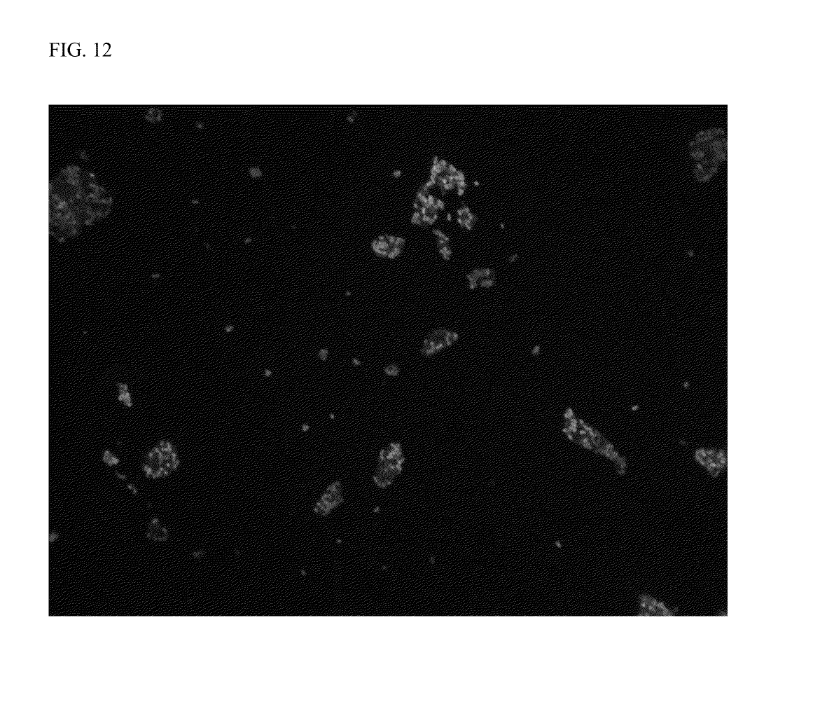

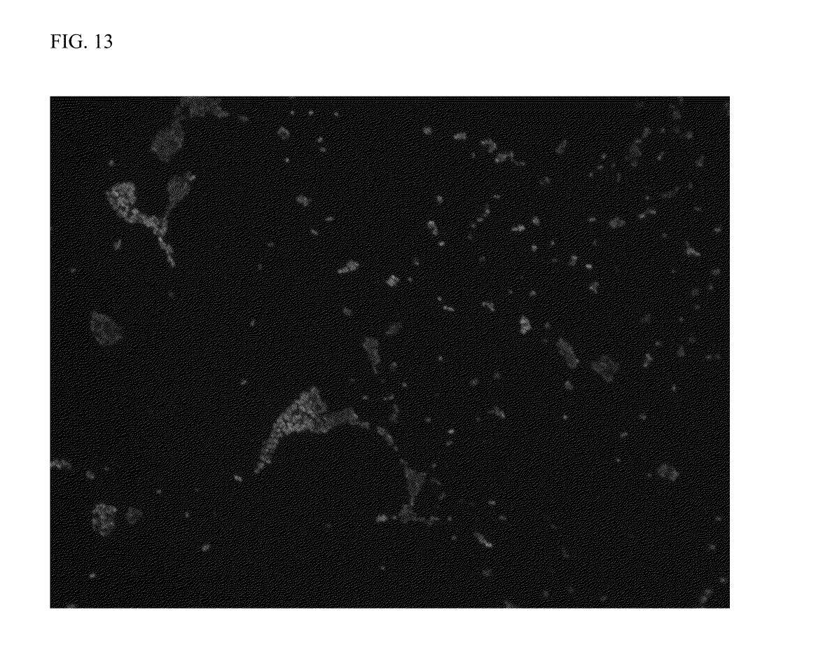

FIG. 10 shows a bright field observation picture of non-immobilized E. coli, observed immediately after preparation of the control solution. Additionally, FIG. 11 shows a fluorescent observation picture of non-immobilized E. coli, observed immediately after preparation of the control solution. In addition, FIG. 12 shows a fluorescent observation picture of the non-immobilized E. coli observed after storage for about one day in a cool dark place after preparation of the control solution, and FIG. 13 shows a fluorescent observation picture of the non-immobilized E. coli observed after storage for about five days in a cool dark place after preparation of the control solution. Note that the images in FIG. 11, FIG. 12, and FIG. 13 are images that were captured with an exposure time of 1.0 seconds.

Dispersive Properties of E. coli with and without Immobilization

When the microscopic observations for the immobilized E. coli, shown in FIG. 6 and FIG. 7, were compared to the microscopic observations of the non-immobilized E. coli, illustrated in FIG. 10 and FIG. 11, it is clear that there are no significant differences in the dispersive characteristics of E. coli depending on whether or not it is immobilized, and that there is essentially no agglomeration caused by the immobilization.

Particle Diameters of the E. coli with and without Immobilization

When the particle diameters of the immobilized E. coli were measured in bright field observation pictures illustrated in FIG. 6, the average particle diameter was 1.5 μm. Moreover, when the particle diameters of the non-immobilized E. coli were measured in bright field observation pictures illustrated in FIG. 10, the average particle diameter was 1.4 μm. Thus the particle diameters for the immobilized E. coli were essentially the same as the particle diameters of the non-immobilized E. coli.

Fluorescent Intensities of E. coli with and without Immobilization

When image analysis software was used to measure the brightness of the image, as fluorescent intensity of the immobilized E. coli, in the fluorescent photograph of the immobilized E. coli photographed with an exposure time of 0.5 seconds immediately after sample preparation, the average fluorescent intensity was 3587.5. When the brightness of the image was measured as the fluorescent intensity of the non-immobilized E. coli, in the fluorescent photograph of the non-immobilized E. coli photographed with an exposure time of 0.5 seconds immediately after control solution preparation, the average fluorescent intensity was 1649.2. The value for the average fluorescent intensity for the immobilized E. coli and the value for the average fluorescent intensity of the non-immobilized E. coli, in the microorganism detecting device were within the range of fluorescent intensities recognized as E. coli.

Attenuation of Fluorescence of E. coli with and without Immobilization

From the comparison of the microorganism photographs of immobilized E. coli, shown in FIG. 7 to FIG. 9, and the microorganism photographs of non-immobilized E. coli, shown in FIG. 11 through FIG. 13, the tendency for fluorescent attenuation of immobilized E. coli is essentially the same as the tendency for fluorescent attenuation of non-immobilized E. coli.

Results of the Observations of the Immobilized S. epidermidis

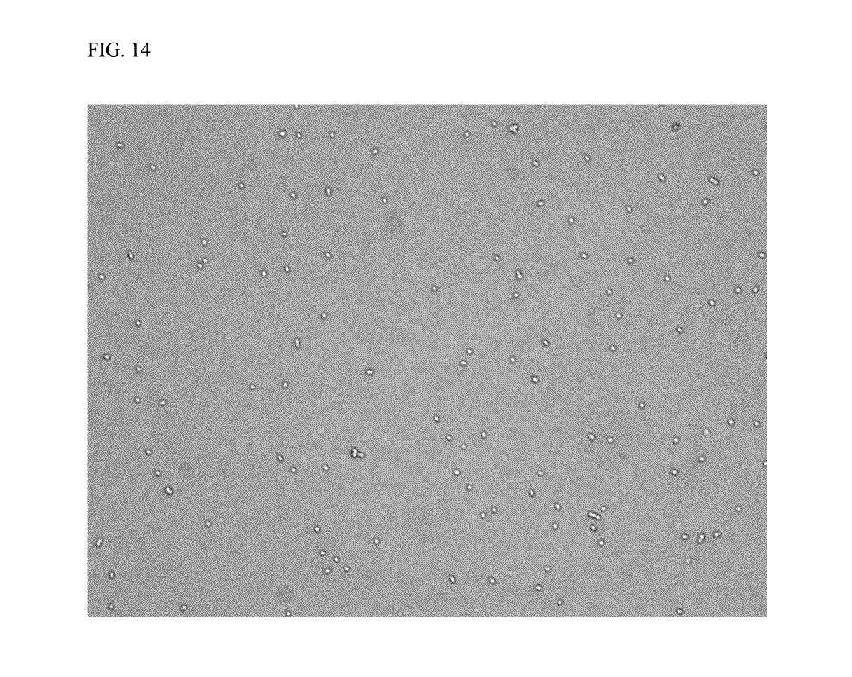

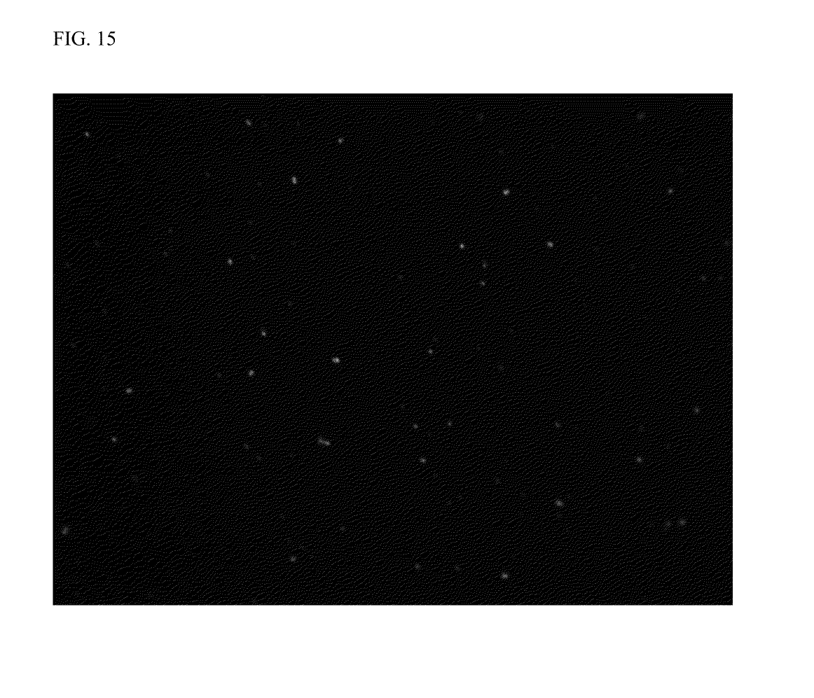

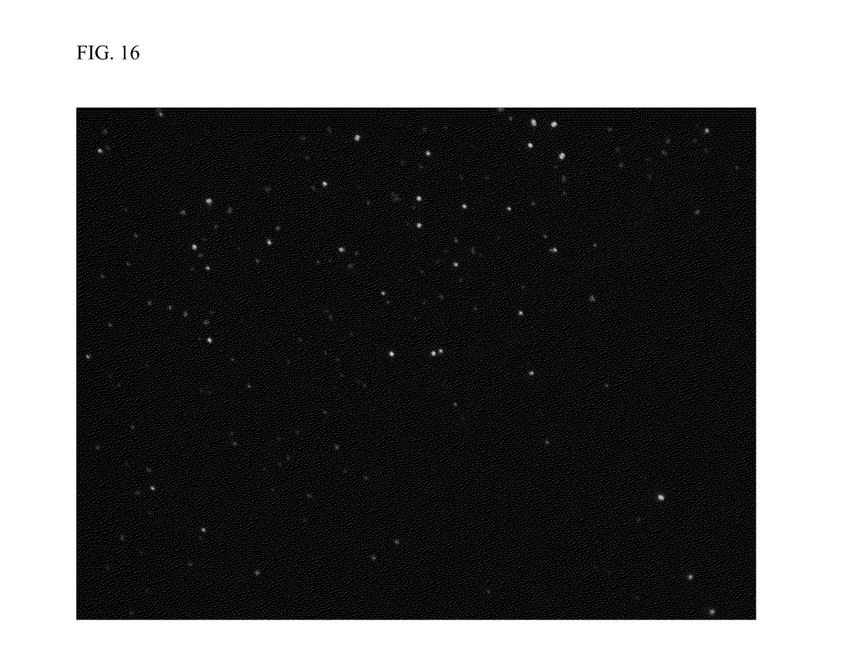

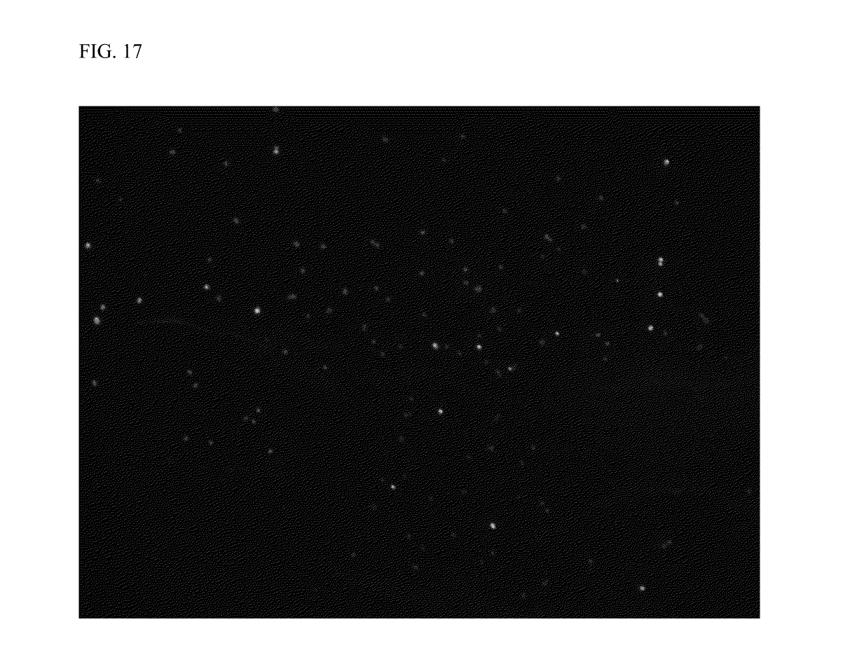

FIG. 14 shows a bright field observation picture of immobilized S. epidermidis, observed immediately after preparation of the sample solution. Additionally, FIG. 15 shows a fluorescent observation picture of immobilized S. epidermidis, observed immediately after preparation of the sample solution. In addition, FIG. 16 shows a fluorescent observation picture of the immobilized S. epidermidis observed after storage for about one day in a cool dark place after preparation of the sample solution, and FIG. 17 shows a fluorescent observation picture of the immobilized S. epidermidis observed after storage for about five days in a cool dark place after preparation of the sample solution. Note that the images in FIG. 15, FIG. 16, and FIG. 17 are images that were captured with an exposure time of 1.0 seconds.

Results of the Observations of the Non-Immobilized S. epidermidis

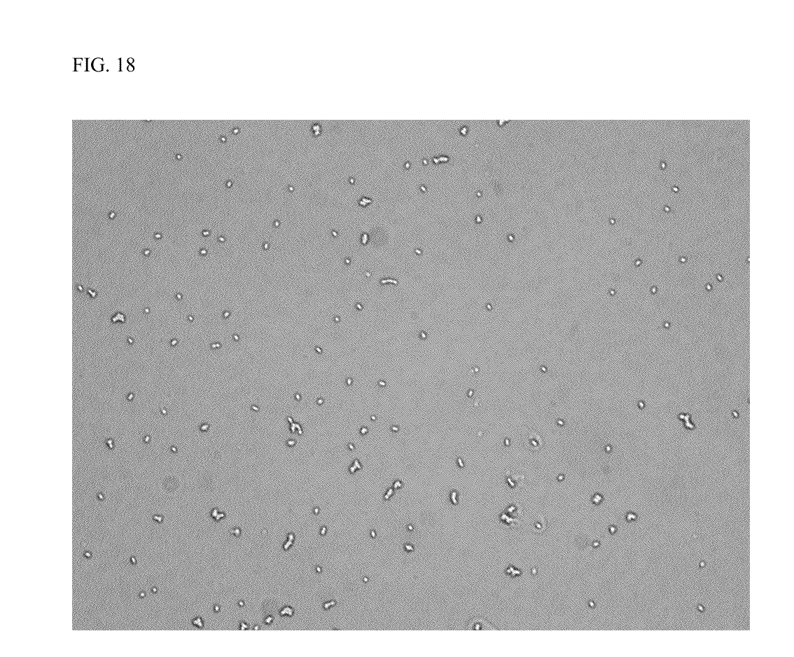

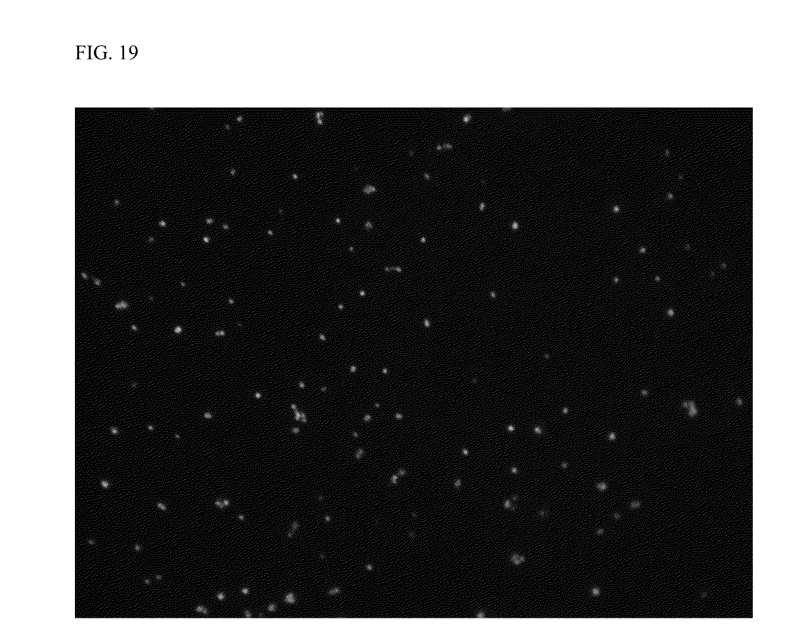

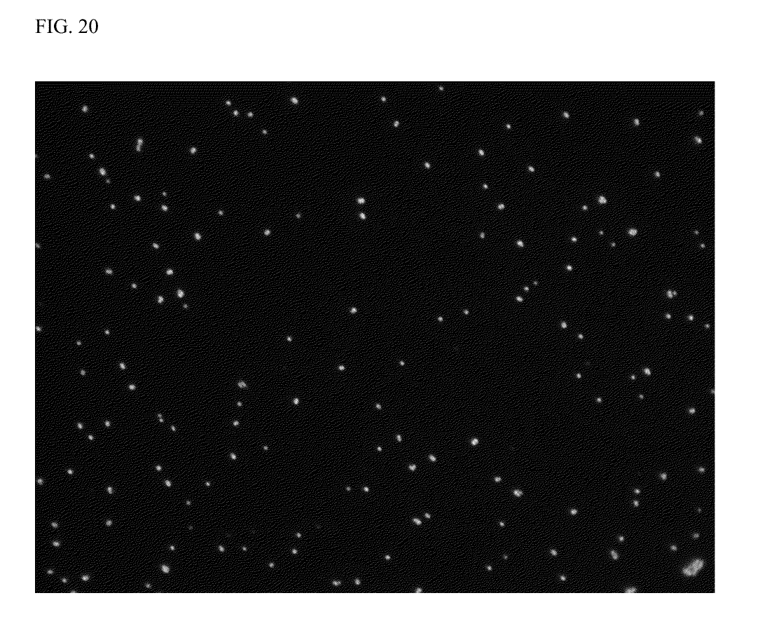

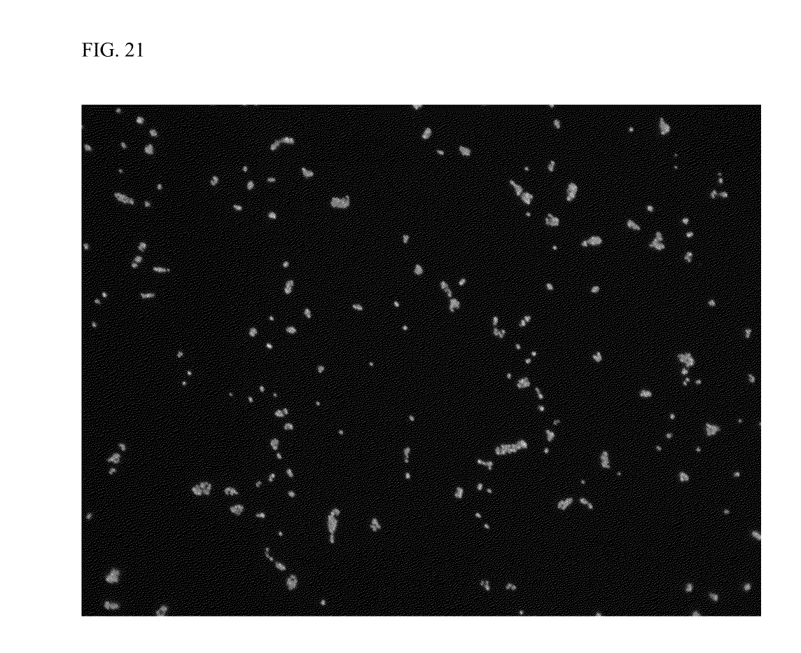

FIG. 18 shows a bright field observation picture of non-immobilized S. epidermidis, observed immediately after preparation of the control solution. Additionally, FIG. 19 shows a fluorescent observation picture of non-immobilized S. epidermidis, observed immediately after preparation of the control solution. In addition, FIG. 20 shows a fluorescent observation picture of the non-immobilized S. epidermidis observed after storage for about one day in a cool dark place after preparation of the control solution, and FIG. 21 shows a fluorescent observation picture of the non-immobilized S. epidermidis observed after storage for about five days in a cool dark place after preparation of the control solution. Note that the images in FIG. 19, FIG. 20, and FIG. 21 are images that were captured with an exposure time of 1.0 seconds.

Dispersive Properties of S. epidermidis with and without Immobilization

When the microscopic observations for the immobilized S. epidermidis, shown in FIG. 14 and FIG. 15, were compared to the microscopic observations of the non-immobilized S. epidermidis, illustrated in FIG. 18 and FIG. 19, it is clear that there are no significant differences in the dispersive characteristics of S. epidermidis depending on whether or not it is immobilized, and that there is essentially no agglomeration caused by the immobilization.

Particle Diameters of S. epidermidis with and without Immobilization

When the particle diameters of the immobilized S. epidermidis were measured in bright field observation pictures illustrated in FIG. 14, the average particle diameter was 1.5 μm. Moreover, when the particle diameters of the non-immobilized S. epidermidis were measured in bright field observation pictures illustrated in FIG. 18, the average particle diameter was 1.6 μm. Thus the particle diameters for the immobilized S. epidermidis were essentially the same as the particle diameters of the non-immobilized S. epidermidis.

Fluorescent Intensities of S. epidermidis with and without Immobilization

When the brightness of the image was measured as the fluorescent intensity of the immobilized S. epidermidis, in the fluorescent photograph of the immobilized S. epidermidis photographed with an exposure time of 0.5 seconds immediately after sample solution preparation, the average fluorescent intensity was 5719.0. Moreover, when the brightness of the image was measured as the fluorescent intensity of the non-immobilized S. epidermidis, in the fluorescent photograph of the non-immobilized S. epidermidis photographed with an exposure time of 0.5 seconds immediately after control solution preparation, the average fluorescent intensity was 6787.8. The value for the average fluorescent intensity for the immobilized S. epidermidis and the value for the average fluorescent intensity of the non-immobilized S. epidermidis, in the microorganism detecting device were within the range of fluorescent intensities recognized as S. epidermidis.

Attenuation of Fluorescence of S. epidermidis with and without Immobilization

From the comparison of the microorganism photographs of immobilized S. epidermidis, shown in FIG. 15 through FIG. 16, and the microorganism photographs of non-immobilized S. epidermidis, shown in FIG. 19 through FIG. 21, the tendency for fluorescent attenuation of immobilized S. epidermidis is essentially the same as the tendency for fluorescent attenuation of non-immobilized S. epidermidis.

Results of the Observations of the Immobilized B. atrophaeus

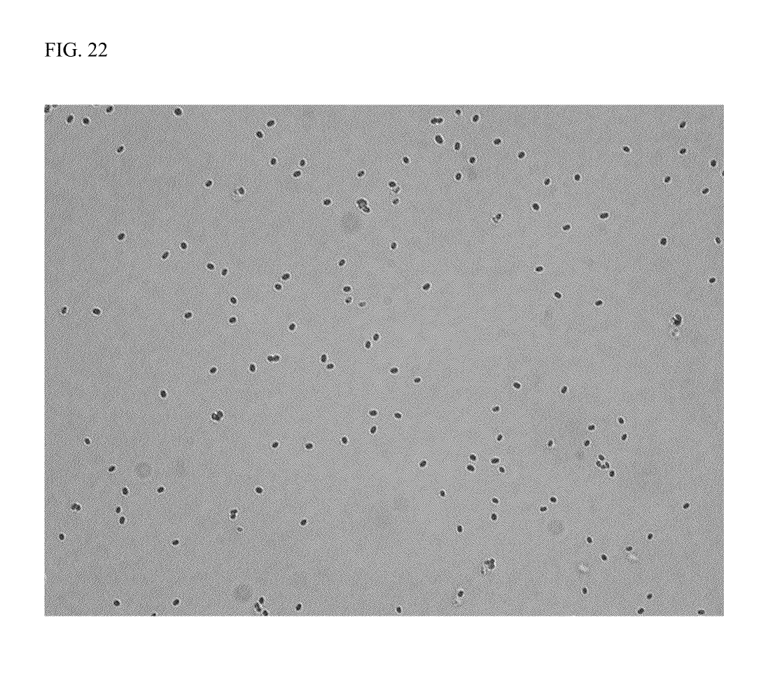

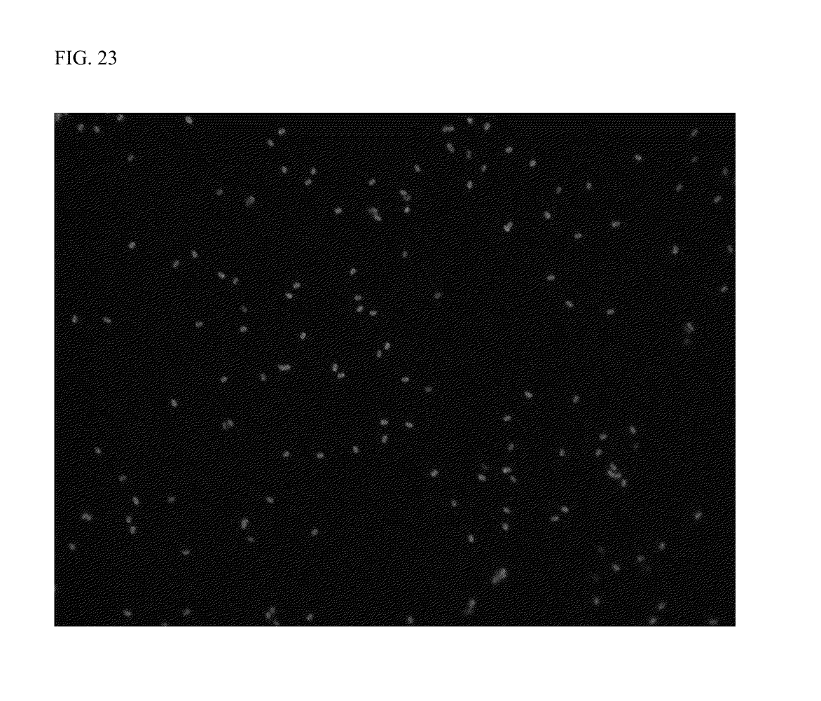

FIG. 22 shows a bright field observation picture of immobilized B. atrophaeus, observed immediately after preparation of the sample solution. Additionally, FIG. 23 shows a fluorescent observation picture of immobilized B. atrophaeus, observed immediately after preparation of the sample solution. Note that the image in FIG. 23 is an image that was captured with an exposure time of 1.0 seconds.

Results of the Observations of the Non-Immobilized B. atrophaeus

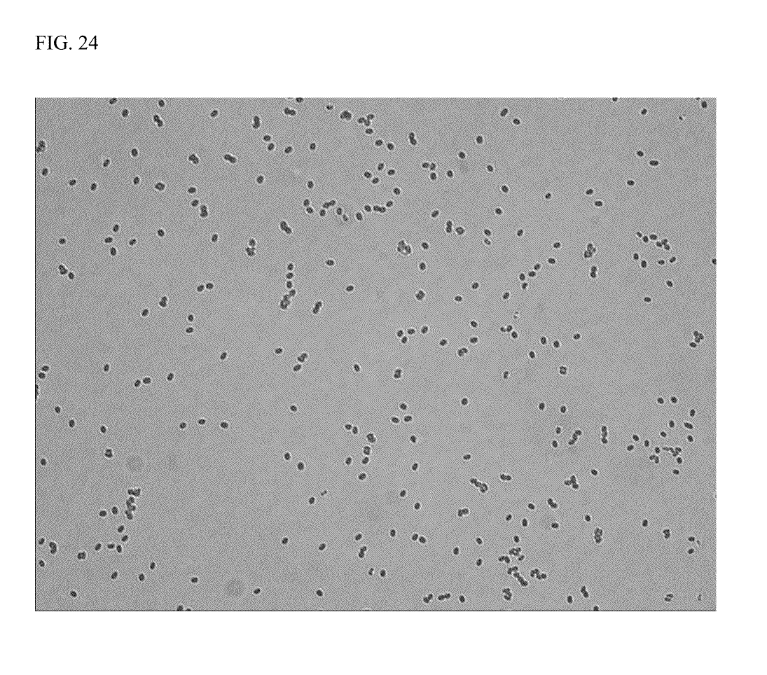

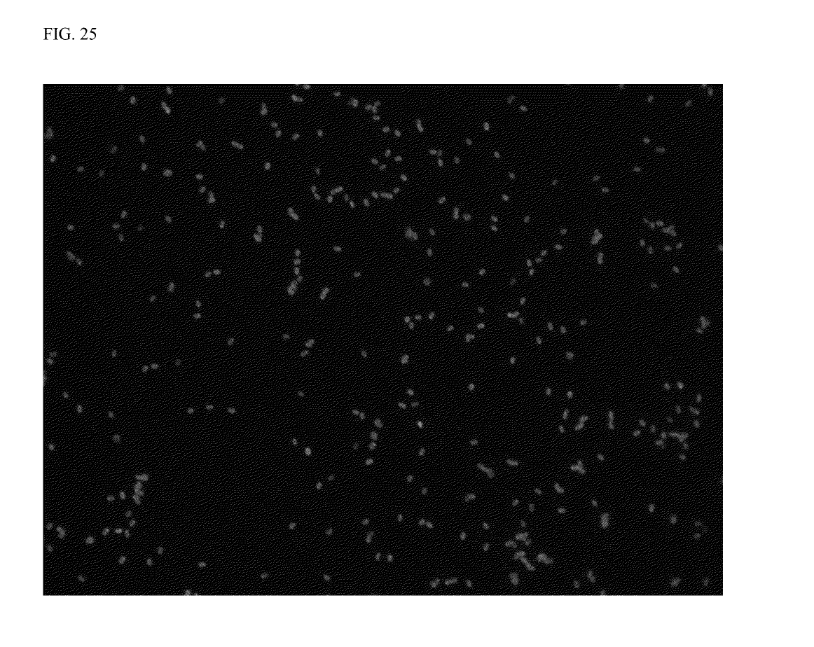

FIG. 24 shows a bright field observation picture of non-immobilized B. atrophaeus, observed immediately after preparation of the control solution. Additionally, FIG. 25 shows a fluorescent observation picture of non-immobilized B. atrophaeus, observed immediately after preparation of the control solution. Note that the image in FIG. 25 is an image that was captured with an exposure time of 1.0 seconds.

Dispersive Properties of B. atrophaeus with and without Immobilization

When the microscopic observations for the immobilized B. atrophaeus, shown in FIG. 22 and FIG. 23, were compared to the microscopic observations of the non-immobilized B. atrophaeus, illustrated in FIG. 24 and FIG. 25, it is clear that there are no significant differences in the dispersive characteristics of B. atrophaeus depending on whether or not it is immobilized, and that there is essentially no agglomeration caused by the immobilization.

Particle Diameters of the B. atrophaeus with and without Immobilization

When the particle diameters of the immobilized B. atrophaeus were measured in bright field observation pictures illustrated in FIG. 22, the average particle diameter was 2.0 μm. Moreover, when the particle diameters of the non-immobilized B. atrophaeus were measured in bright field observation pictures illustrated in FIG. 24, the average particle diameter was 2.0 μm. Thus the particle diameters for the immobilized B. atrophaeus were the same as the particle diameters of the non-immobilized B. atrophaeus.

Fluorescent Intensities of B. atrophaeus with and without Immobilization

When the brightness of the image was measured as the fluorescent intensity of the immobilized B. atrophaeus, in the fluorescent photograph of the immobilized B. atrophaeus photographed with an exposure time of 0.5 seconds immediately after sample solution preparation, the average fluorescent intensity was 9182.0. When the brightness of the image was measured as the fluorescent intensity of the non-immobilized B. atrophaeus, in the fluorescent photograph of the non-immobilized B. atrophaeus photographed with an exposure time of 0.5 seconds immediately after control solution preparation, the average fluorescent intensity was 7264.0. The value for the average fluorescent intensity for the immobilized B. atrophaeus and the value for the average fluorescent intensity of the non-immobilized B. atrophaeus, in the microorganism detecting device were within the range of fluorescent intensities recognized as B. atrophaeus.

Results of the Observations of the Immobilized A. niger

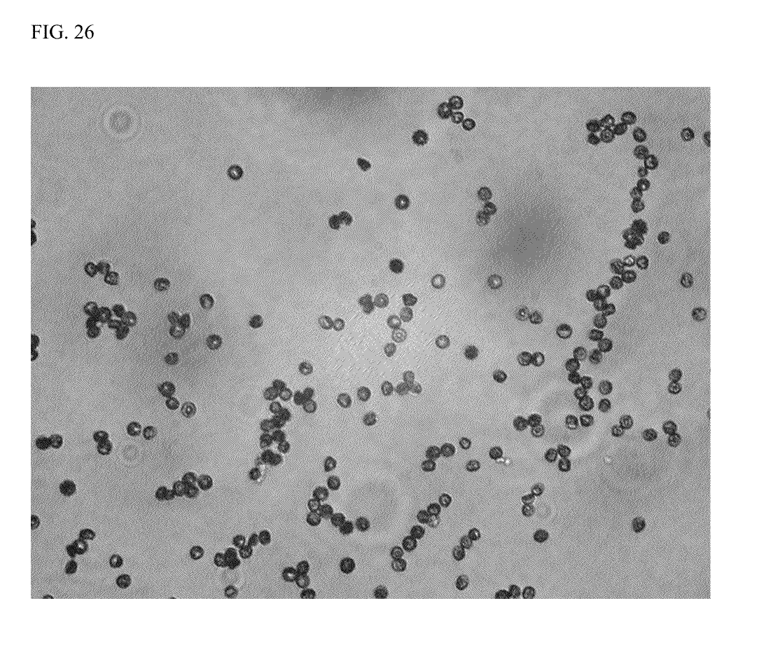

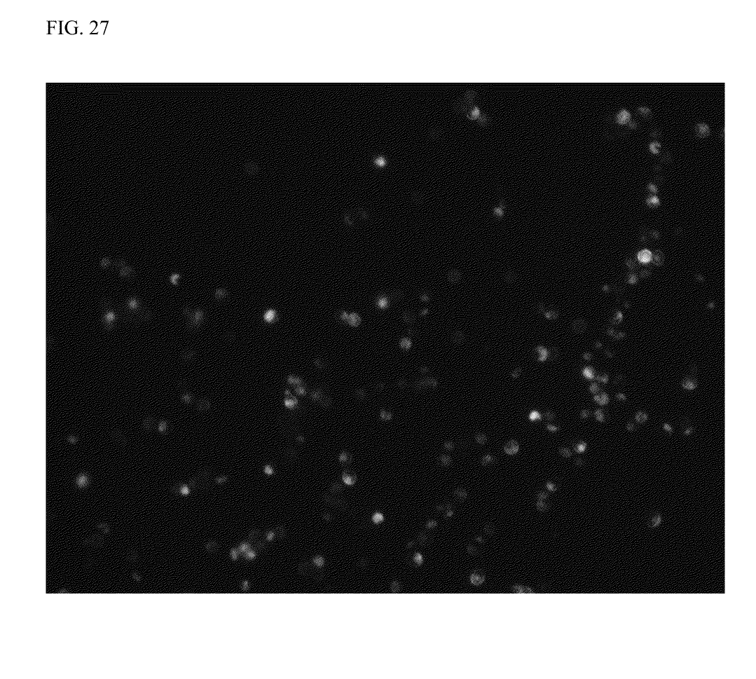

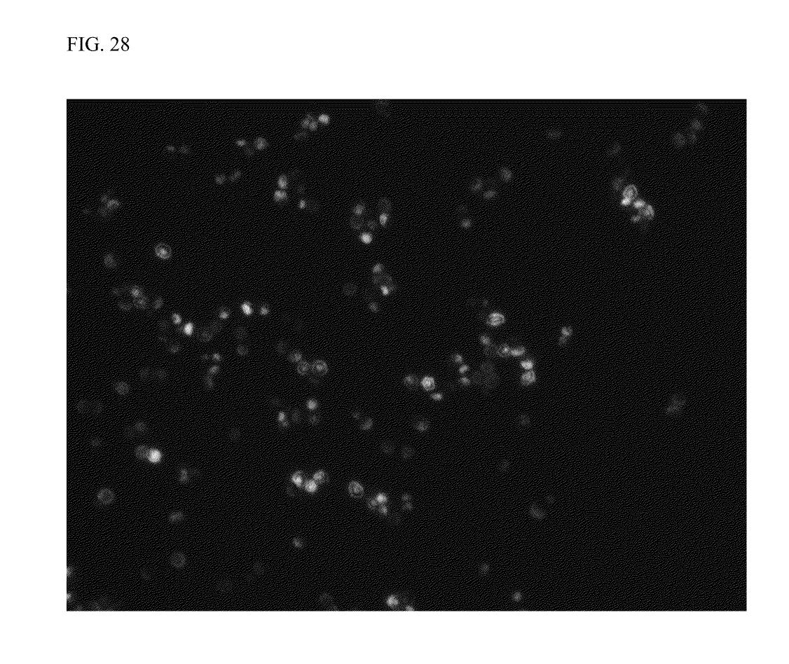

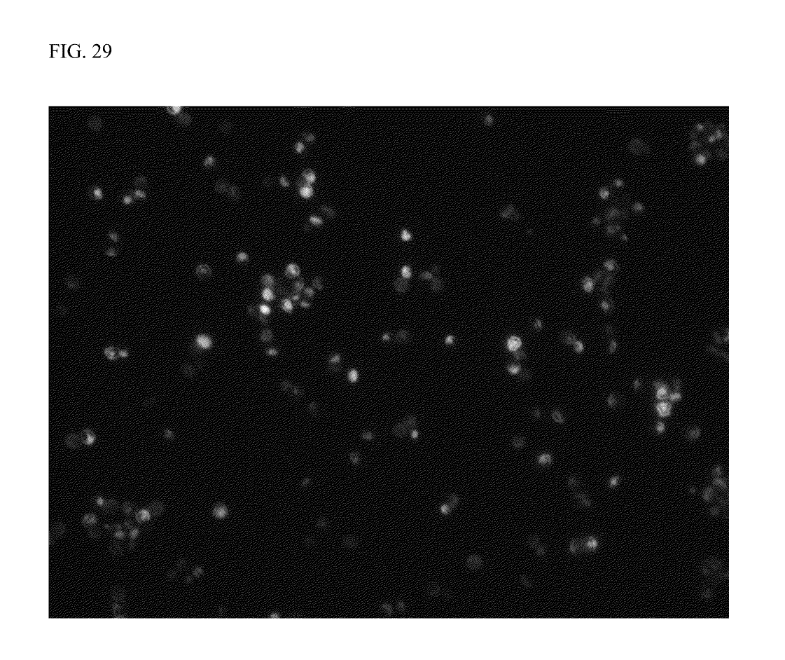

FIG. 26 shows a bright field observation picture of immobilized A. niger. Additionally, FIG. 27 shows a fluorescent observation picture of immobilized B. atrophaeus, observed immediately after preparation of the sample solution. In addition, FIG. 28 shows a fluorescent observation picture of the immobilized A. niger observed after storage for about one day in a cool dark place after preparation of the sample solution, and FIG. 29 shows a fluorescent observation picture of the immobilized A. niger observed after storage for about five days in a cool dark place after preparation of the sample solution. Note that the images in FIG. 27, FIG. 28, and FIG. 29 are images that were captured with an exposure time of 1.0 seconds.

Results of the Observations of the Non-Immobilized A. niger

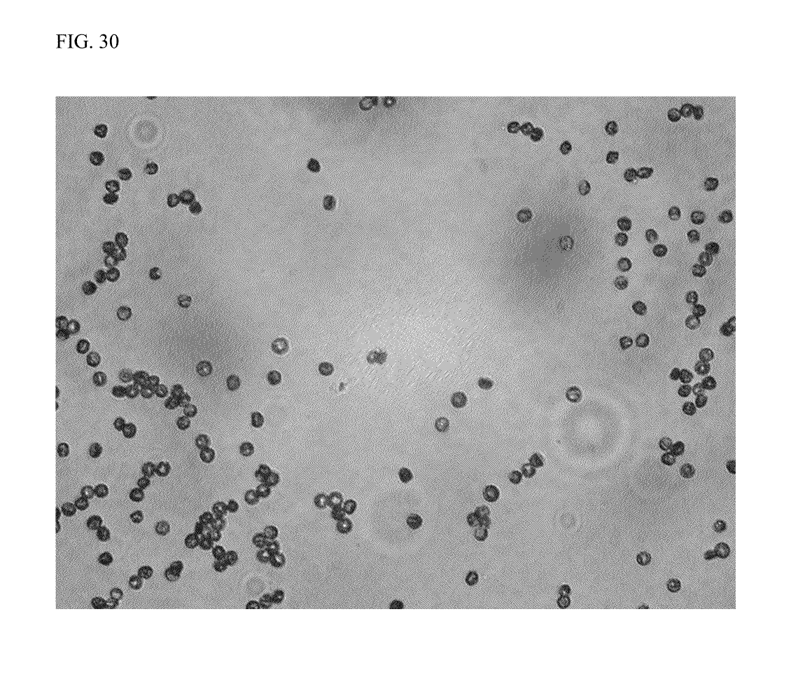

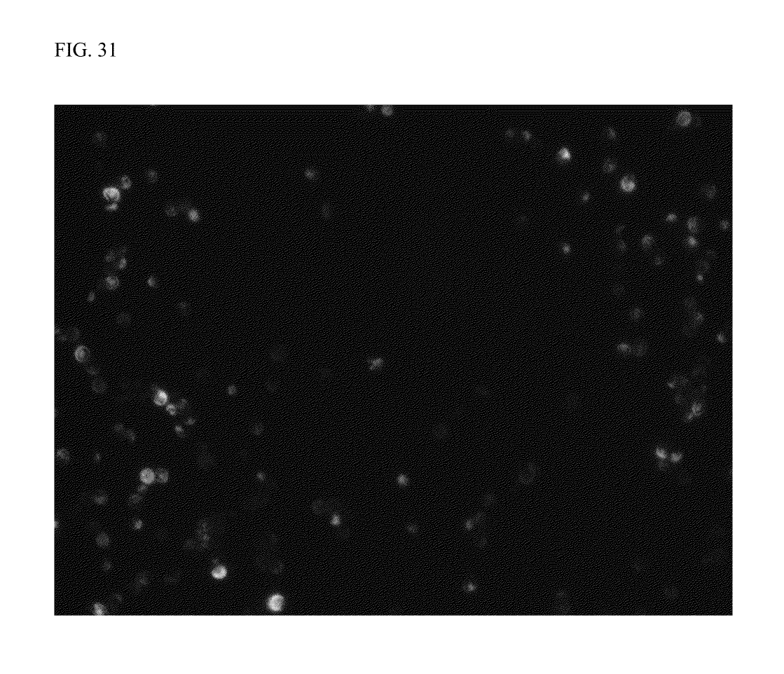

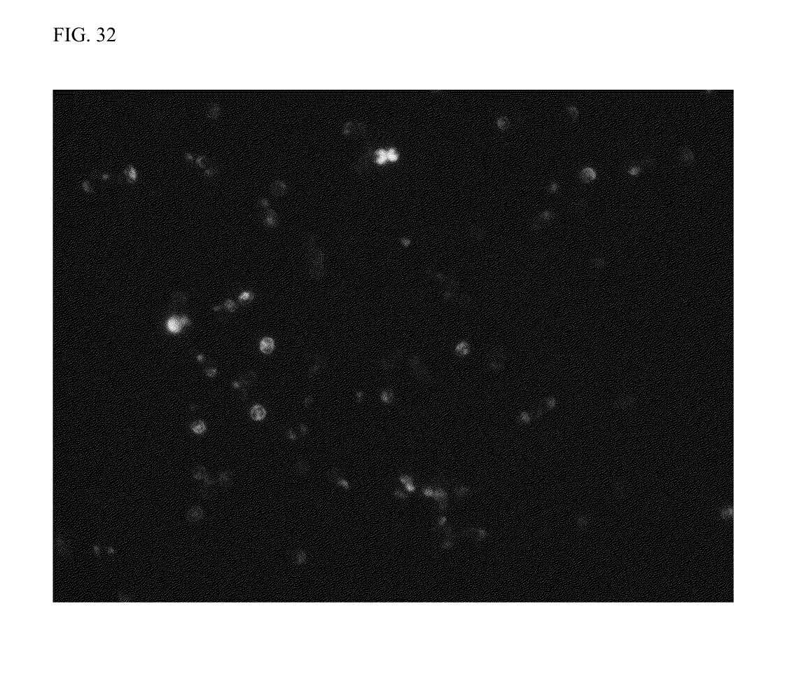

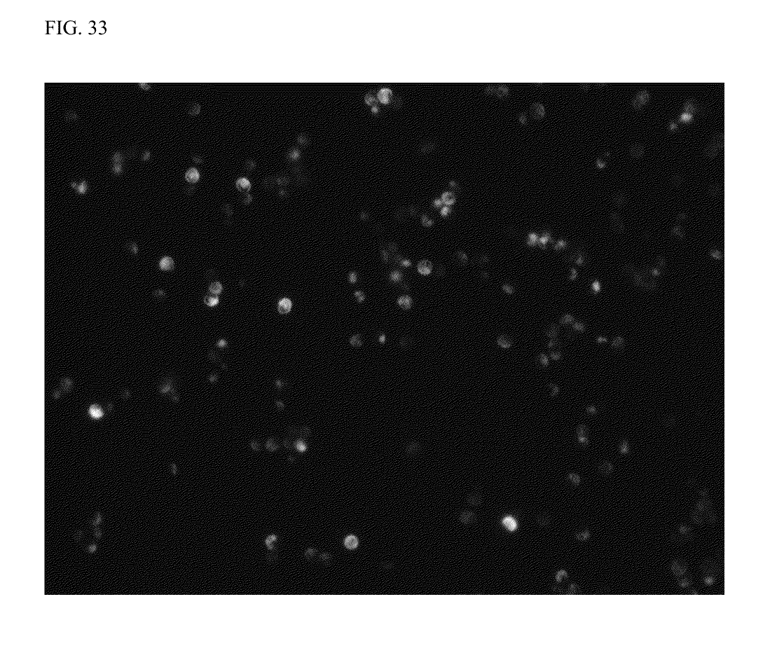

FIG. 30 shows a bright field observation picture of non-immobilized A. niger, observed immediately after preparation of the control solution. Additionally, FIG. 31 shows a fluorescent observation picture of non-immobilized B. atrophaeus, observed immediately after preparation of the control solution. In addition, FIG. 32 shows a fluorescent observation picture of the non-immobilized A. niger observed after storage for about one day in a cool dark place after preparation of the control solution, and FIG. 33 shows a fluorescent observation picture of the non-immobilized A. niger observed after storage for about five days in a cool dark place after preparation of the control solution. Note that the images in FIG. 31, FIG. 32, and FIG. 33 are images that were captured with an exposure time of 1.0 seconds.

Dispersive Properties of A. niger with and without Immobilization

When the microscopic observations for the immobilized A. niger, shown in FIG. 26 and FIG. 27, were compared to the microscopic observations of the non-immobilized A. niger, illustrated in FIG. 30 and FIG. 31, it is clear that there are no significant differences in the dispersive characteristics of A. niger depending on whether or not it is immobilized, and that there is essentially no agglomeration caused by the immobilization.

Particle Diameters of the A. niger with and without Immobilization

When the particle diameters of the immobilized A. niger were measured in bright field observation pictures illustrated in FIG. 26, the average particle diameter was 3.2 μm. Moreover, when the particle diameters of the non-immobilized A. niger were measured in bright field observation pictures illustrated in FIG. 30, the average particle diameter was 3.5 μm. Thus the particle diameters for the immobilized A. niger were essentially the same as the particle diameters of the non-immobilized A. niger.

Fluorescent Intensities of A. niger with and without Immobilization

When the brightness of the image was measured as the fluorescent intensity of the immobilized A. niger, in the fluorescent photograph of the immobilized A. niger photographed with an exposure time of 0.5 seconds immediately after sample solution preparation, the average fluorescent intensity was 18900.3. When the brightness of the image was measured as the fluorescent intensity of the non-immobilized A. niger, in the fluorescent photograph of the non-immobilized A. niger photographed with an exposure time of 0.5 seconds immediately after control solution preparation, the average fluorescent intensity was 17342.1. The value for the average fluorescent intensity for the immobilized A. niger and the value for the average fluorescent intensity of the non-immobilized A. niger, in the microorganism detecting device were within the range of fluorescent intensities recognized as A. niger.

Attenuation of Fluorescence of A. niger with and without Immobilization

From the comparison of the microorganism photographs of immobilized A. niger, shown in FIG. 27 to FIG. 29, and the microorganism photographs of non-immobilized A. niger, shown in FIG. 31 through FIG. 33, the tendency for fluorescent attenuation of immobilized A. niger is essentially the same as the tendency for fluorescent attenuation of non-immobilized A. niger.

Results of the Observations of the Immobilized P. putida

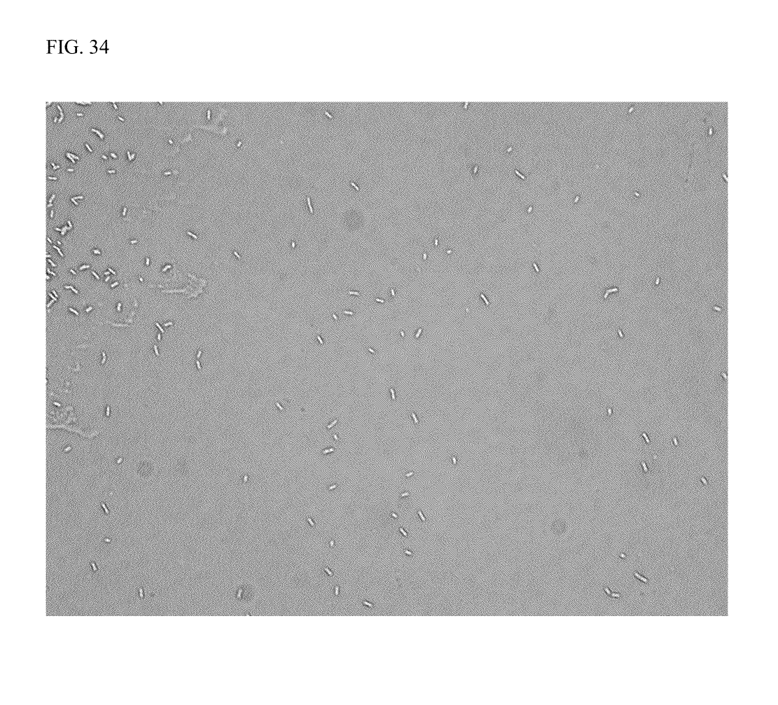

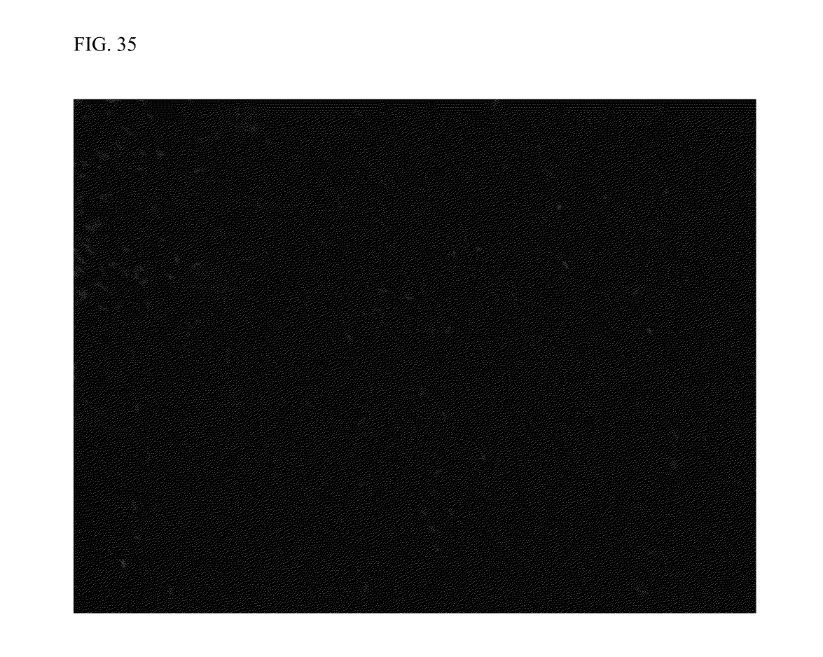

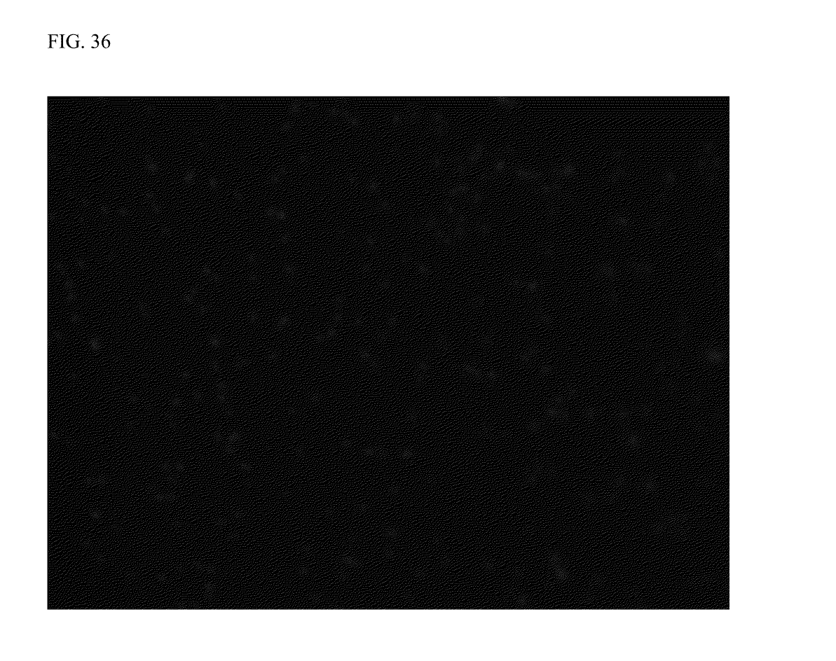

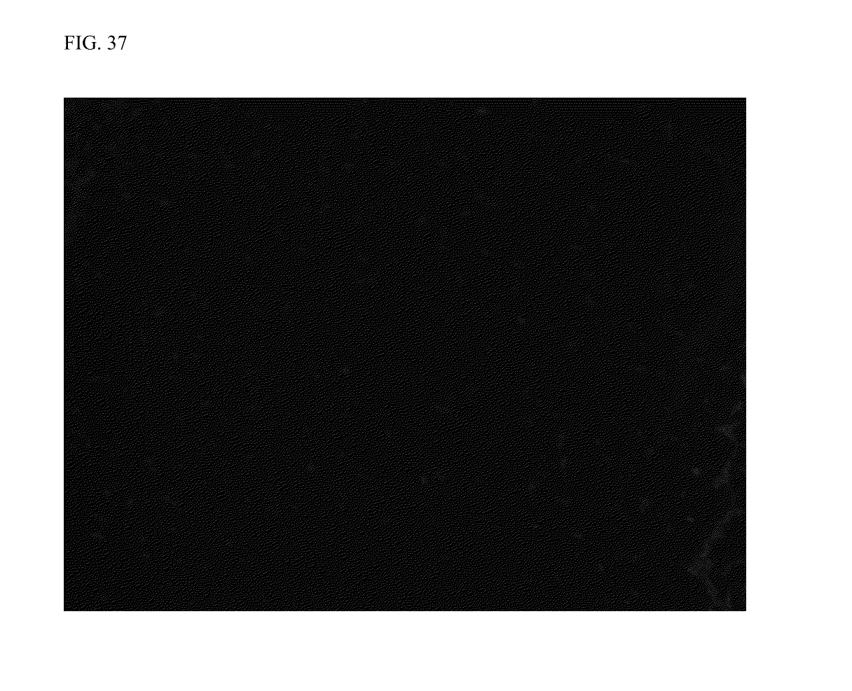

FIG. 34 shows a bright field observation picture of immobilized P. putida, observed immediately after preparation of the sample solution. Additionally, FIG. 35 shows a fluorescent observation picture of immobilized P. putida, observed immediately after preparation of the sample solution. In addition, FIG. 36 shows a fluorescent observation picture of the immobilized P. putida observed after storage for about one day in a cool dark place after preparation of the sample solution, and FIG. 37 shows a fluorescent observation picture of the immobilized P. putida observed after storage for about five days in a cool dark place after preparation of the sample solution. Note that the images in FIG. 35, FIG. 36, and FIG. 37 are images that were captured with an exposure time of 1.0 seconds.

Results of the Observations of the Non-Immobilized P. putida

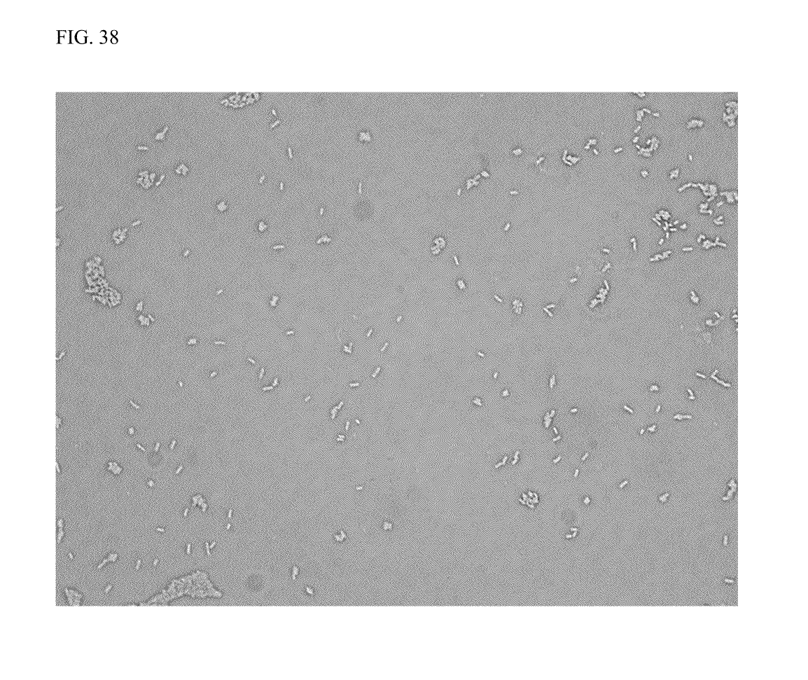

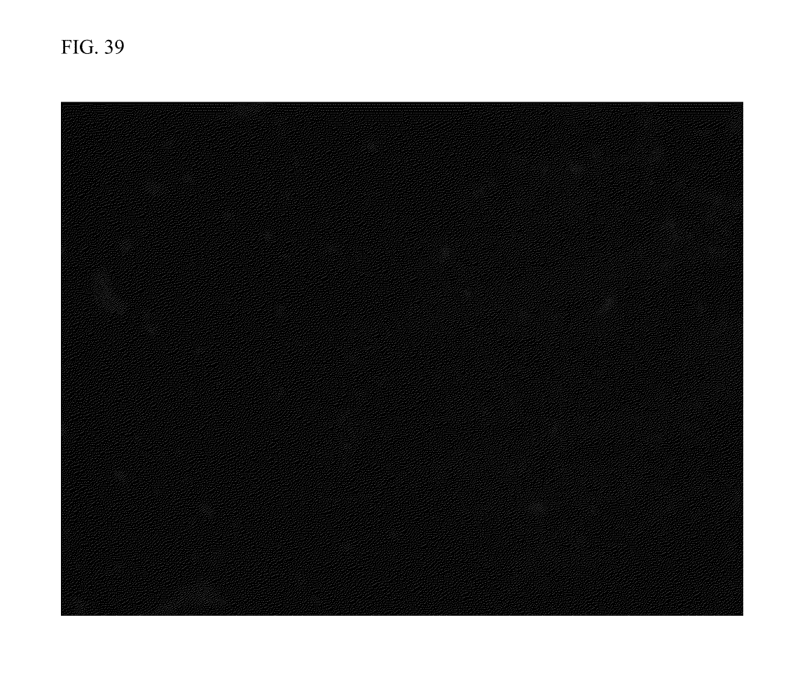

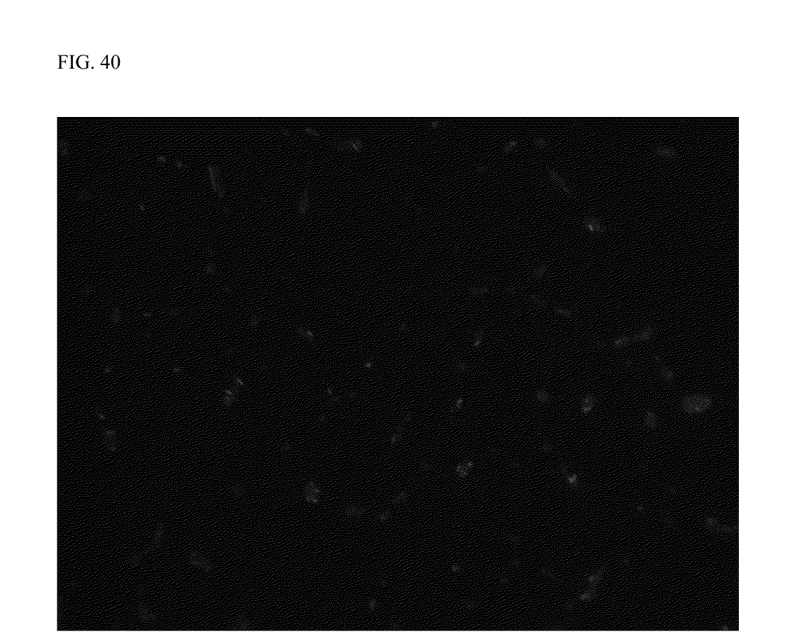

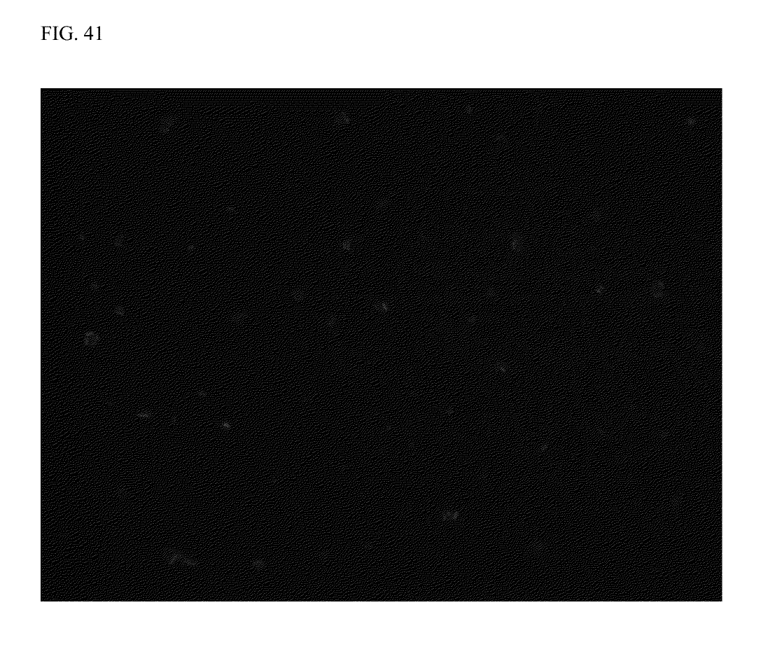

FIG. 38 shows a bright field observation picture of non-immobilized P. putida, observed immediately after preparation of the control solution. Additionally, FIG. 39 shows a fluorescent observation picture of non-immobilized P. putida, observed immediately after preparation of the control solution. In addition, FIG. 40 shows a fluorescent observation picture of the non-immobilized P. putida observed after storage for about one day in a cool dark place after preparation of the control solution, and FIG. 41 shows a fluorescent observation picture of the non-immobilized P. putida observed after storage for about five days in a cool dark place after preparation of the control solution. Note that the images in FIG. 39, FIG. 40, and FIG. 41 are images that were captured with an exposure time of 1.0 seconds.

Dispersive Properties of P. putida with and without Immobilization

When the microscopic observations for the immobilized P. putida, shown in FIG. 34 and FIG. 35, were compared to the microscopic observations of the non-immobilized P. putida, illustrated in FIG. 38 and FIG. 39, it is clear that there are no significant differences in the dispersive characteristics of P. putida depending on whether or not it is immobilized, and that there is essentially no agglomeration caused by the immobilization.

Particle Diameters of P. putida with and without Immobilization

When the particle diameters of the immobilized P. putida were measured in bright field observation pictures illustrated in FIG. 34, the average particle diameter was 0.7 μm. Moreover, when the particle diameters of the non-immobilized P. putida were measured in bright field observation pictures illustrated in FIG. 38, the average particle diameter was 0.6 μm. Thus the particle diameters for the immobilized P. putida were essentially the same as the particle diameters of the non-immobilized P. putida. Note that because P. putida is bacilliform, the diameter when converted into a circle with the same surface area was calculated as the diameter.

Fluorescent Intensities of P. putida with and without Immobilization

When image analysis software was used to measure the brightness of the image, as fluorescent intensity of the immobilized P. putida, in the fluorescent photograph of the immobilized P. putida photographed with an exposure time of 0.5 seconds immediately after sample preparation, the average fluorescent intensity was 694.9. When the brightness of the image was measured as the fluorescent intensity of the non-immobilized P. putida, in the fluorescent photograph of the non-immobilized P. putida photographed with an exposure time of 0.5 seconds immediately after control solution preparation, the average fluorescent intensity was 265.9. The value for the average fluorescent intensity for the immobilized P. putida and the value for the average fluorescent intensity of the non-immobilized P. putida, in the microorganism detecting device were within the range of fluorescent intensities recognized as P. putida.

Attenuation of Fluorescence of P. putida with and without Immobilization

From the comparison of the microorganism photographs of immobilized P. putida, shown in FIG. 35 to FIG. 37, and the microorganism photographs of non-immobilized P. putida, shown in FIG. 39 through FIG. 41, the tendency for fluorescent attenuation of immobilized P. putida is essentially the same as the tendency for fluorescent attenuation of non-immobilized P. putida.

Properties of Microorganisms with and without Immobilization

The average fluorescent intensity of standard fluorescent particles observed simultaneously with the E. coli for which the fluorescent intensity was measured was 128324.1. The standardized average fluorescent intensity of the immobilized E. coli, which is the average fluorescent intensity of the immobilized E. coli divided by the average fluorescent intensity of the standard fluorescent particles, was 0.027956. The standardized average fluorescent intensity of the non-immobilized E. coli, which is the average fluorescent intensity of the non-immobilized E. coli divided by the average fluorescent intensity of the standard fluorescent particles, was 0.012852.

The average fluorescent intensity of standard fluorescent particles observed simultaneously with the S. epidermidis for which the fluorescent intensity was measured was 125189.1. The standardized average fluorescent intensity of the immobilized S. epidermidis, which is the average fluorescent intensity of the immobilized S. epidermidis divided by the average fluorescent intensity of the standard fluorescent particles, was 0.045683. The standardized average fluorescent intensity of the non-immobilized S. epidermidis, which is the average fluorescent intensity of the non-immobilized S. epidermidis divided by the average fluorescent intensity of the standard fluorescent particles, was 0.05422.

The average fluorescent intensity of standard fluorescent particles observed simultaneously with the B. atrophaeus for which the fluorescent intensity was measured was 126602.2. The standardized average fluorescent intensity of the immobilized B. atrophaeus, which is the average fluorescent intensity of the immobilized B. atrophaeus divided by the average fluorescent intensity of the standard fluorescent particles, was 0.072527. The standardized average fluorescent intensity of the non-immobilized B. atrophaeus, which is the average fluorescent intensity of the non-immobilized B. atrophaeus divided by the average fluorescent intensity of the standard fluorescent particles, was 0.057377.

The average fluorescent intensity of standard fluorescent particles observed simultaneously with the A. niger for which the fluorescent intensity was measured was 141894.6. The standardized average fluorescent intensity of the immobilized A. niger, which is the average fluorescent intensity of the immobilized A. niger divided by the average fluorescent intensity of the standard fluorescent particles, was 0.133199. The standardized average fluorescent intensity of the non-immobilized A. niger, which is the average fluorescent intensity of the non-immobilized A. niger divided by the average fluorescent intensity of the standard fluorescent particles, was 0.122218.

The average fluorescent intensity of standard fluorescent particles observed simultaneously with the P. putida for which the fluorescent intensity was measured was 124202.9. The standardized average fluorescent intensity of the immobilized P. putida, which is the average fluorescent intensity of the immobilized P. putida divided by the average fluorescent intensity of the standard fluorescent particles, was 0.005595. The standardized average fluorescent intensity of the non-immobilized P. putida, which is the average fluorescent intensity of the non-immobilized P. putida divided by the average fluorescent intensity of the standard fluorescent particles, was 0.002141.

Because there is variability in the measurement results for the fluorescent intensities emitted by the fluorescent substances depending on the state of the microscope equipment, there is variability depending on the date and time of the observation. Because of this, as described above, the variability of the fluorescent intensity due to fluorescent observations of the microorganisms at different dates and times were eliminated through measuring fluorescent intensities of reference fluorescent particles of the same types simultaneously with the respective microorganisms, and then dividing the fluorescent intensities of the microorganisms by the fluorescent intensities of the standard fluorescent particles.

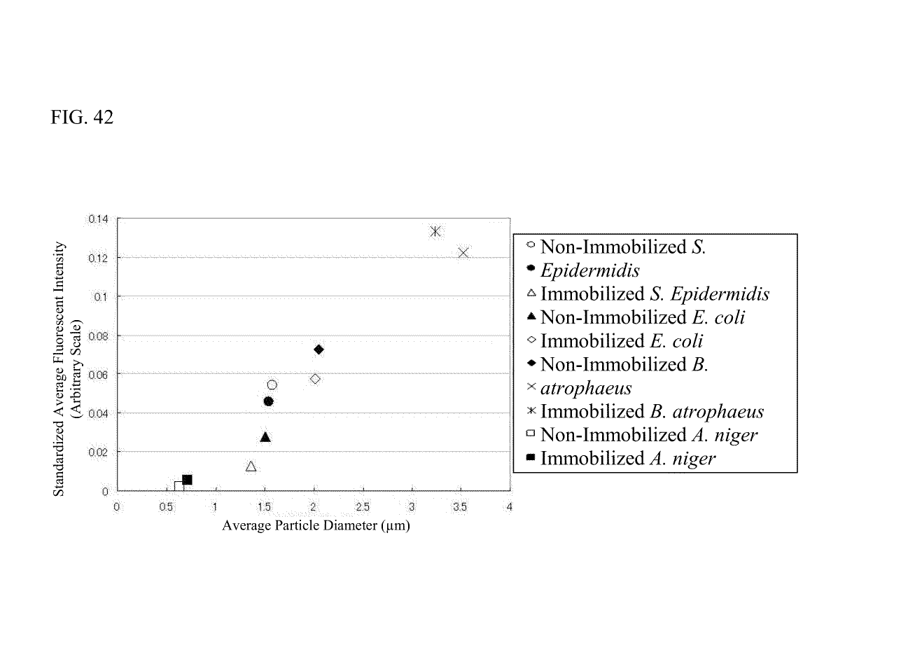

When the particle diameters and the standardized average fluorescent intensities were plotted for each of the immobilized microorganisms and non-immobilized microorganisms, the characteristics of the microorganisms exhibited the identical trends, with regardless of whether or not immobilization was performed, as illustrated in FIG. 42. Moreover, when the variability of the characteristics of the immobilized and non-immobilized microorganisms were compared, they were small when compared to the differences in characteristics between different types of microorganisms.

### Other Examples

While there are descriptions of the examples as set forth above, the descriptions and drawings that form a portion of the disclosure are not to be understood to limit the present invention. A variety of alternate examples and operating technologies should be obvious to those skilled in the art. For example the principal for detecting microorganisms in a microorganism detecting device that is evaluated through the immobilized microorganisms in the present example is not limited to that which is described above. The immobilized microorganisms according to the example may be used to evaluate a microorganism detecting device that detects microorganisms through the principle of electrophoresis, such as induced electrophoresis, or to evaluate a microorganism detecting device for detecting microorganisms through the principle of flow cytometry instead. In this way, the present invention should be understood to include a variety of examples, and the like, not set forth herein.

1. A microorganism detecting device evaluation kit, comprising:
an immobilized microorganism.

2. The microorganism detecting device evaluation kit as set forth in claim 1, wherein:
the immobilized microorganism is dispersed in a solvent.

3. The microorganism detecting device evaluation kit as set forth in claim 2, wherein:
the solvent is water.

4. The microorganism detecting device evaluation kit as set forth in claim 1, wherein:
the immobilized microorganism is immobilized in an aldehyde.

5. The microorganism detecting device evaluation kit as set forth in claim 4, wherein:
the aldehyde is formaldehyde.

6. The microorganism detecting device evaluation kit as set forth in claim 1, wherein:
the immobilized microorganism emits fluorescent light.

7. A microorganism detecting device evaluation kit manufacturing method, comprising:
immobilizing a microorganism.

8. The microorganism detecting device evaluation kit manufacturing method as set forth in claim 7, comprising:
dispersing the immobilized microorganism in a solvent.

9. The microorganism detecting device evaluation kit manufacturing method as set forth in claim 8, wherein:
the solvent is water.

10. The microorganism detecting device evaluation kit manufacturing method as set forth in claim 7, wherein:
an aldehyde is used in the immobilizing.

11. The microorganism detecting device evaluation kit manufacturing method as set forth in claim 10, wherein:
the aldehyde is formaldehyde.

12. The microorganism detecting device evaluation kit manufacturing method as set forth in claim 7, wherein:
the immobilized microorganism emits fluorescent light.

13. A microorganism detecting device evaluating method comprising:
preparing an immobilized microorganism; and
detecting the immobilized microorganism with a microorganism detecting device.

14. The microorganism detecting device evaluating method as set forth in claim 13, wherein:
the detecting the immobilized microorganism includes:
illuminating the immobilized microorganism with light; and
receiving fluorescent light that is produced by the immobilized microorganism.

15. The microorganism detecting device evaluating method as set forth in claim 13, wherein:
the detecting the immobilized microorganism includes:
illuminating the immobilized microorganism with light; and
receiving scattered light that is produced by the immobilized microorganism.

16. The microorganism detecting device evaluating method as set forth in claim 13, wherein:
the immobilized microorganism is dispersed in a solvent.

17. The microorganism detecting device evaluating method as set forth in claim 16, wherein:
an aerosol is produced from the solution wherein the immobilized microorganism is dispersed in a solvent; and
the aerosol is introduced into the microorganism detecting device.

18. The microorganism detecting device evaluating method as set forth in claim 16, wherein:
the solvent is water.

19. The microorganism detecting device evaluating method as set forth in claim 13, wherein:
the immobilized microorganism is immobilized in an aldehyde.

20. The microorganism detecting device evaluating method as set forth in claim 19, wherein:
the aldehyde is formaldehyde.
